(19) United States
(12) Patent Application Publication
(10) Pub. No.: US 2008/0305097 A1
(43) Pub. Date: Dec. 11, 2008

(54) Use of Protease or a Protease Inhibitor for the Manufacture of Medicaments

(75) Inventors: Tsvee Lapidot, Ness Ziona (IL); Orit Kollet, Ramat Gan (IL)

Correspondence Address: MARSHALL, GERSTEIN & BORUN LLP, 233 S. WACKER DRIVE, SUITE 6300, SEARS TOWER, CHICAGO, IL, 60606, US

(73) Assignee: YEDA RESEARCH AND DEVELOPMENT CO., LTD., Rehovot (IL)

(21) Appl. No.: 11/576,064

(22) PCT Filed: Oct. 27, 2005
(86) PCT No.: PCT/IL05/01121

§ 371 Date: Jul. 22, 2008

(30) Foreign Application Priority Data
Oct. 31, 2004 (IL) 164942

(51) Int. Cl. A61K 38/43; C12Q 1/48; A61P 31/00

(52) U.S. Cl. 424/94.5; 435/15

(57) ABSTRACT

The invention relates to the use of a cathepsin K inhibitor (CTKI) or CTK, a mutein, isoform, fused protein, functional derivative, active fraction, circularly permutated derivative, a salt or inducer thereof in the manufacture of a medicament for treating a disease in which SDF-1 activity and/or concentration is involved with the development and/or course of the disease.

### FIELD OF THE INVENTION

The present invention relates to the use of cathepsin K (CTK) or a cathepsin K inhibitor (CTKI) in the manufacture of a medicament for treating a disease characterized by the involvement of SDF-1 with the development and/or course of the disease.

### BACKGROUND OF THE INVENTION

The morphologically recognizable and functionally capable cells circulating in blood include erythrocytes, neutrophilic, eosinophilic, and basophilic granulocytes, B-, T-, non B-, non T-lymphocytes, and platelets. These mature hematopoietic cells derive from and are replaced, on demand, by morphologically recognizable dividing precursor cells for the respective lineages such as erythroblasts for the erythrocytes series, myeloblasts, promyelocytes and myelocytes for the granulocyte series, and megakaryocytes for the platelets. The precursor cells arise from more primitive cells that can be simplistically divided into two major subgroups: stem cells and progenitor cells [for review, see (1)}.

CXCL12 or stromal cell-derived 1 factor (SDF-1) is a potent chemoattractant for resting lymphocytes, monocytes, and CD34-positive hematopoietic progenitor cells (2). SDF-1 binds only to one receptor, CXCR4, which has only this chemokine as known ligand.

SDF-1 at low concentrations, leads to the activation of leukocyte integrins, the arrest of leukocytes on capillary endothelial cells, and extravasation of these cells (3). SDF-1 controls B-cell lymphopoiesis and bone marrow myelopoiesis (4) and is critical for bone marrow engraftment (13). It promotes CD4+ T-cell survival and primes these cells for cytokine and T-cell receptor-mediated stimuli (5). It was shown that the functional activity of the SDF-1/CXCR4 complex can be inhibited in vitro by proteolysis mediated by leukocyte elastase and this could be a mechanism by which the homing of hematopoietic cell progenitors is regulated (6). In fact, the egress of CD34+ hematopoietic cell progenitors from the bone marrow and other organs to the peripheral blood (mobilization) induced by the granulocyte-colony stimulating factor (G-CSF) in vivo relies on the proteolytic inactivation of SDF-1 by neutrophil elastase (41).

It is known that Cathepsin G and elastase cleave SDF-1 in G-CSF induced mobilization as demonstrated by Levesque and Petit.

Two growth factors (cytokines), granulocyte colony-stimulating factor (G-CSF) and granulocyte-macrophage colony-stimulating factor (GM-CSF), are widely used to prevent fever and infections in patients with severe neutropenia (eg, after bone marrow transplantation and intensive cancer chemotherapy). Cytokine therapy is expensive; however, if the risk of febrile neutropenia is >=30%, the cost of G-CSF is justified. In general, most clinical benefit occurs when G-CSF is administered about 24 h after the completion of chemotherapy. Doses of 5 μg/kg/day sc are often effective. G-CSF and GM-CSF accelerate the return of the neutrophil count to >500/μL in patients undergoing autologous bone marrow transplantation or intensive chemotherapy.

Neutrophil production in congenital, cyclic, and idiopathic neutropenia can be improved by administration of G-CSF 3 to 10 μg/kg/day sc. This therapy is indicated in patients free from mouth ulcers and other types of oropharyngeal inflammation, fever, and cellulitis and other documented bacterial infections. The benefits are sustained, and patients can be maintained on daily or alternate-day G-CSF for months or years without loss of its effectiveness. Long-term G-CSF has also been used to induce neutrophil generation and neutrophil mobilization to prevent neutropenia in other circumstances, including myelodysplasia, HIV and AIDS, and autoimmune disorders. In general, neutrophil counts increase, although clinical benefits of this therapy are less clear, especially for patients who do not have severe neutropenia. Patients with neutropenia caused by an idiosyncratic drug reaction may also benefit from G-CSF, particularly if a delayed recovery is anticipated. Thus far, however, only uncontrolled trials have been reported in the latter situation.

Interactions between SDF-1, and its receptor CXCR4 play an essential role in stem cell seeding of the BM during murine embryonic development (10,11). Previously, the present inventors were able to show, using immune deficient NOD/SCID mice as recipients, that both the short term in vivo migration (homing) and high-level multilineage repopulation of the murine bone marrow by human CD34+ enriched cells are dependent on SDF-1/CXCR4 interactions (12-15). In support of these data, it has been shown that either high levels of CXCR4 expression on human CD34+ cells, or high SDF-1 induced directional motility in vitro, correlates with faster recovery in both allogeneic and autologous clinical transplantations with positive selection of CD34+ cells (16,17).

It is well documented that low concentrations of SDF-1 in synergy with other early acting cytokines enhance proliferation of both human CD34+ cells and murine stem and progenitor cells, suggesting a role for this chemokine in progenitor cell survival (25-29), while high levels of SDF-1 induce quiescence of proliferating human long term culture initiating cells (LTCIC) and primitive human fetal liver CD34+ stem cells capable of serial repopulation of transplanted NOD/SCID mice (30,31).

CXCR4 expression is a dynamic process, which is regulated by environmental factors such as cytokines, chemokines, stromal cells, adhesion molecules, and proteolytic enzymes (18). In hematopoietic stem and progenitor cells of human origin, CXCR4 can be upregulated from intracellular pools by short term (˜40 hr) in vitro cytokine culture (13,19) or stimulation of cord blood (CB) CD34+ with proteolytic enzymes such as MMP-2 and MMP-920. This subsequently enhances their in vitro migration towards an SDF-1 gradient (13) as well as their in vivo homing and repopulation capacities in transplanted NOD/SCID and serially transplanted b2mnull NOD/SCID mice (12,13), linking stem cell self renewal and development with motility. A recent report demonstrated that longer culture periods with a cytokine cocktail results in a decrease in cell surface CXCR4 expression on human CB CD34+ enriched cells (22) and reduced repopulation was documented with human progenitors cultured in vitro for longer periods (23). Recently, the present inventors showed that CB CD34+/CXCR4− sorted cells harbor low levels of intracellular CXCR4, which, following short term in vitro cytokine stimulation, can rapidly be functionally expressed on the cell surface to mediate SDF-1 dependent homing and repopulation of transplanted NOD/SCID mice (15).

Proteolitic cleavage associated with inactivation of SDF-1 was demonstrated by several degrading enzymes including MMP-2 and MMP-9, Cathepsin-G and elastase (Petit et al. Natl Immunol. 3:687 2002, Valenzuela-Femandez et al. JBC 277:15677, 2002, Levesque t al. JCI 111:187, 2003 and McQuibban et al. JBC 276: 43503, 2001).

In addition to their central role in mediating directional migration of human and murine stem cells 24, SDF-1/CXCR4 interactions are also involved in other stem cell functions. Of importance, SDF-1/CXCR4 interactions are also involved in retention of stem and progenitor cells in the BM (10,32,33). This hypothesis has also been confirmed by other studies which demonstrated the involvement of SDF-1/CXCR4 interactions in the anchorage of human hematopoietic stem cells (HSC) injected directly into the murine BM cavity (34,35). Interference of these interactions induces release/mobilization of both human and murine progenitors from the BM into the circulation (36-41).

Bone is undergoing a constant remodeling process that is balanced through the activities of bone-generating osteoblasts and bone-resorbing osteoclasts. Various bone diseases, such as osteoporosis, Paget's disease, certain forms of arthritis, and osseous metastases are characterized by excessive osteoclast-mediated bone resorption. RANKL (Receptor Activator of NK-kappaB Ligand) and its corresponding RANK receptor play a crucial role in osteoclast differentiation and activation, and osteoprotegerin (OPG) is the physiological inhibitor of RANKL.

Matrix degradation is mainly due to the activity of the cysteine protease, CTK (7, 8). CTK is a lysosomal cysteine protease that is highly expressed in osteoclasts and is implicated in bone resorption (a process by which osteoclasts degrade bone). Type I collagen constitutes 90-95% of the organic bone mass (Krane, S. M., and Simon, L. (1994) in Scientific American Medicine (Rubenstein, E., and Federman, D. D., eds), Vol. 3, pp. 1-26, Scientific American, Inc., New York) and represents the major biological substrate for CTK (9). Among all mammalian collagenases, CTK is the only protease capable of cleaving interstitial collagens at multiple sites within their triple helical structures (Garnero, P., Borel, O., Byrjalsen, I., Ferreras, M., Drake, F. H., McQueney, M. S., Foged, N. T., Delmas, P. D., and Delaisse, J. M. (1998) J. Biol. Chem. 273, 32347-32352, 6 Kafienah, W., Bromme, D., Buttle, D. J., Croucher, L. J., and Hollander, A. P. (1998) Biochem. J. 331, 727-732). Deficiency in CTK activity leads to an accumulation of undigested collagen fibrils in lysosomes within osteoclasts (Everts, V., Aronson, D.C., and Beertsen, W. (1985) Calcif. Tissue Int. 37, 25-31), as observed in patients with the autosomal recessive skeletal dysplasia pycnodysostosis (Gelb, B. D., Shi, G. P., Chapman, H. A., and Desnick, R. J. (1996) Science 273, 1236-1238). Analysis of CTK mutants revealed that the collagen degradation defect is not necessarily coupled with the loss of proteolytic activity of CTK. One disease causing mutation, Y212C, that is remote from the active site of the protease, only mildly affected the overall proteolytic activity of CTK but completely eliminated its collagenase activity (Hou, W.-S., Brömme, D., Zhao, Y., Mehler, E., Dushey, C., Weinstein, H., Miranda, C. S., Fraga, C., Greig, F., Carey, J., Rimoin, D. L., Desnick, R. J., and Gelb, B. D. (1999) J. Clin. Invest. 103, 731-738). This observation indicated that in addition to the catalytic activity of CTK, other features are required for the hydrolysis of collagens by CTK. Bone- and cartilage-resident glycosaminoglycans specifically enhance the degradation of interstitial collagens of types I and II by CTK, an effect not observed with cathepsin L or matrix metalloproteinase I (Li, Z., Hou, W. S., and Bromme, D. (2000) Biochemistry 39, 529-536). This finding suggested that the collagenase activity of CTK requires specific interactions between CTK protein and glycosaminoglycans. The mechanism by which CTK degrades collagen, however, remained elusive. Li Z, et al (J Biol Chem. 2002 Aug. 9; 277(32):28669-76. Epub 2002 May 30) demonstrated that the collagenolytic activity of CTK depends on the formation of a novel oligomeric complex of CTK protein with chondroitin sulfate. Thus currently, CTK inhibitors are being used for inhibiting bone reabsortion.

### SUMMARY OF THE INVENTION

The invention relates the use of a cathepsin K inhibitor (CTKI) or CTK, a mutein, isoform, fused protein, functional derivative, active fraction, circularly permutated derivative, a salt or inducer thereof in the manufacture of a medicament for treating a disease in which SDF-1 activity and/or concentration is involved with the development and/or course of the disease.

In one aspect of the invention, CTK is used in a disease is caused/aggravated by SDF-1 activity, such as cancer, inflammation and infection.

In one embodiment of the invention, the disease is allergic airway disease.

In a further embodiment of the invention, the disease is rheumatoid arthritis.

In a further embodiment of the invention, the disease is arteriosclerosis.

In a further embodiment of the invention, the disease is cancer such as prostate cancer, kidney cancer, neuroblastoma, glioma, pancreatic cancer, colon cancer, breast cancer, leukemia such as acute lymphoblastic leukemia (ALL) and Acute Myeloid Leukemia (AML).

In a further embodiment of the invention, CTK is used in for preventing cancer metastasis, preferably in cancer cells expressing CXCR4

In another aspect of the invention CTKI is used in a disease is prevented/alleviated by SDF-1 activity such as HIV.

In addition, the invention provides the use of a CTK, a mutein, isoform, fused protein, functional derivative, active fraction, circularly permutated derivative, a salt or inducer thereof in the manufacture of a medicament for inducing mobilization of stem cells in a subject in need.

In one embodiment, the subject in need suffers from severe neutropenia, for example occurring following bone marrow transplantation or cancer chemotherapy.

Also, the invention provides the use of a CTKI in the manufacture of a medicament for increasing retention of stem cells in the bone marrow in a subject in need, for example for enhancing repopulation of an organ such as bone marrow, liver and kidney, in a subject in need.

The invention also provides a method of treating a disease which its development and course is affected by SDF-1 activity and/or concentration, comprising administration of an effective amount of CTKI or a CTK, a mutein, isoform, fused protein, functional derivative, active fraction, circularly permutated derivative, a salt or inducer thereof.

In another aspect, the invention provides a method of treating cancer in a mammal, comprising administering to the mammal an effective amount of CTK, a mutein, isoform, fused protein, functional derivative, active fraction, circularly permutated derivative, a salt or inducer thereof.

In a preferred embodiment of the invention, the cancer cell expresses CXC Chemokine Receptor-4 (CXCR4).

In another preferred embodiment, the method of the invention is used for preventing metastasis.

In addition, the invention provides a method of modulating targeting of pluripotent stem cells to tissues comprising the administration of an effective amount of a CTKI or CTK, a mutein, isoform, fused protein, functional derivative, active fraction, circularly permutated derivative, a salt or inducer thereof in a subject in need.

In one embodiment of the invention, CTKI is administered to a target tissue for increasing targeting of cells, such as normal hematopoietic cells, to the target tissue.

In a preferred embodiment of the invention, the cells are in vivo in a patient and a therapeutically effective amount of the CXCR4 agonist, such as SDF-1, a mutein, isoform, fused protein, functional derivative, active fraction, circularly permutated derivative, a salt or inducer thereof, is administered to the patient in need such as a cancer patient.

In another preferred embodiment, the patient requires autologous or allogeneic bone marrow or peripheral blood stem cell transplantation and/or the patient is treated with a cytotoxic agent.

In another embodiment, the invention provides a method in which CTK, a mutein, isoform, fused protein, functional derivative, active fraction, circularly permutated derivative, a salt or inducer thereof is administered to a target tissue for reducing targeting of the cells, for example neoplastic cells, to the target tissue.

In another aspect, the invention provides a method of reducing the rate of hematopoietic cell multiplication, comprising administering an effective amount of a CTK, a mutein, isoform, fused protein, functional derivative, active fraction, circularly permutated derivative, a salt or inducer thereof to the hematopoietic cells.

In one embodiment of the invention, the hematopoietic cells is selected from the group consisting of hematopoietic stem cells and hematopoietic progenitor cells.

In a further embodiment of the invention, the cells are in vivo in a patient, and a therapeutically effective amount of the CXCR4 agonist, such as SDF-1, a mutein, isoform, fused protein, functional derivative, active fraction, circularly permutated derivative, a salt or inducer thereof is administered to the patient in need.

In a further embodiment of the invention, the patient requires autologous or allogeneic bone marrow or peripheral blood stem cell transplantation.

In a further embodiment of the invention, the patient has a cancer and is treated with a cytotoxic agent.

In another aspect, the invention provides a method of identifying a CTK antagonist comprising contacting CTK with SDF-1, measuring the activity of SDF-1 and isolating a compound capable of preventing or reversing inhibition of SDF-1 activity by CTK.

The invention provides also a method of identifying a CTK antagonist comprising contacting CTK with SDF-1, checking the integrity of SDF-1 and isolating a compound capable of preventing the degradation SDF-1 activity by CTK.

The invention relates to the antagonist obtained by the method of the invention.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows that LPS induced inflammation leads to osteoclast activation. Balb/c mice were IP injected with a single injection of LPS (250 mcg/mouse). Five days later bones were harvested and activated osteoclasts were detected by enzymatic TRAP staining, in red (A). BM was harvested and seeded in the presence of 20 ng/ml of mouse M-SCF and soluble mouse RANKL, which induce osteoclast differentiation and activation, an assay used to quantify the levels of osteoclast BM precursors (B). The expression of SDF-1, HGF and cathepsin K (CTK) was assessed by RT-PCR (C).

FIG. 2 shows that LPS-induced inflammation leads to SDF-1 degradation and progenitor mobilization. BM fluids and peripheral blood cells were collected from Mice described in FIG. 1. (A) SDF-1 concentration in BM fluids, determined by ELISA. (B) number of colonies raised by PB progenitors, assayed in semi solid culture.

FIG. 3 shows that LPS induced inflammation leads to reduced SDF-1 and cell egress from the BM within 16 h. Mice treated with a single injection of LPS (250 mcg) were killed 16 h later. BM SDF-1 concentration was determined by ELISA (A) and numbers of mononuclear cells in the BM and blood circulation was determined by hemacytometer (B).

FIG. 4 shows that SDF-1/CXCR4 interactions are involved in LPS-induced mobilization of progenitors. 16 h post a single injection of LPS (250 mcg), Balb/c mice were sacrificed, and BM and PB were harvested. CXCR4 expression was measured by flow cytometry (A) and the level of progenitors in the circulation was assayed in semi solid cultures (B).

FIG. 5 shows that functional CXCR4 and MMP 2/9 are required for LPS-mediated mobilization. Balb/c mice were injected with a single injection of LPS (250 mcg). MMP 2/9 inhibitor (100 mcg) and anti CXCR4 (10 ug) were injected twice: 2 h before and 2 h after LPS injection. 16 h after LPS injection, BM and PB were harvested and the number of leukocytes in the Blood (A) and BM (B) were evaluated using hemocytometer.

FIG. 6 shows reduced levels of SDF-1 and increased CXCR4 expression in response to stress signals induced by controlled bleeding. Balb/c mice were treated by a single bleeding of 1% of body weight. After 3, 7, 10 and 14 days, BM and PB were harvested. SDF-1 levels in the BM were measured by ELISA (A). CXCR4 expression by PB cells was quantified by flow cytometry (B) and the levels of circulating progenitors which indicate mobilization, was assayed in semi solid cultures (C).

FIG. 7 shows that controlled bleeding induce osteoclast activation. Femurs of mice described in FIG. 6, were fixed, decalcified, paraffin embedded and sectioned. TRAP staining was used to detect activated osteoclasts in red.

FIG. 8 shows that in vitro stimulation with G-CSF, HGF and SDF-1 induce Ocl activation. Murine primary calvaria osteoblasts were grown in the presence of Vitamine D3 and PG2E to potentiate the secretion of M-CSF and expression of RANKL. BM cells were seeded on the osteoblast monolayer without or with G-CSF (50 ng/ml), HGF (50 ng/ml), SDF-1 (10 and 100 ng/ml). Mature activated osteoclasts were detected 5 days later by TRAP staining. (A) representative TRAP staining. (B) summary of A.

FIG. 9 shows increased SDF-1 secretion by primary murine osteoblasts in response to G-CSF and HGF. Mouse primary calvaria osteoblasts were cultured for 3 days in the presence of 50 ng/ml G-CSF and HGF. (A) Expression of SDF-1 mRNA, assayed by RT-PCR. (B) levels of SDF-1 secreted by treated osteoblasts, assayed by ELISA.

FIG. 10 shows that the cytokines SDF-1 and HGF induce osteoclast activation and HPC mobilization in vivo. Balb/c mice were injected with 5 daily injections of SDF-1 (10 mcg) and HGF (1.5 mcg). Bones of treated mice were stained for TRAP indicating for mature active osteoclasts in red (A). Peripheral blood cells were assayed for the levels of circulating progenitors by semi solid cultures, indicating for mobilization (B).

FIG. 11 shows that the Ocl differentiating factor RANKL induces in vivo formation of TRAP+ active osteoclasts along the endosteum. Balb/c mice were injected with soluble murine RANKL (2 injections per day, 5 mcg per subcutaneous injection). Bones of treated mice were stained for TRAP (in red) indicating mature activated osteoclasts (A). Levels of MMP-9 in BM fluids were determined by zymography assay (B). The expression of CTK mRNA was detected by RT-PCR (C).

FIG. 12 shows that RANKL treatment induces progenitor mobilization, mediated by CXCR4 and MMP2/9. (A) Progenitor mobilization assayed in semisolid cultures, induced by SDF-1 (10 mcg), HGF (1.5 mcg) injected for 5 days and sRANKL (2×5 mcg) injected for the first 3 days. (B) RANKL induced mobilization is inhibited by administration of anti CXCR4 antibodies (10 mcg) and MMP2/9 inhibitor (100 mcg), injected for the last 2 days. (C) Osteoclast precursors in the BM of treated mice were assayed by culturing BM cells for 4 days with M-CSF and sRANKL, followed by TRAP staining to detect mature active osteoclasts.

FIG. 13 shows that chemotactic activity of SDF-1 is abolished by CTK, the major osteoclast bone resorbing enzyme. SDF-1 (125 ng) was incubated with 1 mcg/ml CTK (final concentration in the reaction) for 16 h, 37OC. Protease inhibitor was incubated with CTK for 1.5 h before SDF-1 was added. Migration of Pre B ALL G2 cells towards the treated SDF-1 indicates its chemotactic activity.

FIG. 14 shows that SDF-1 is N-terminally cleaved by CTK. SDF-1 (20 ng) was incubated with CTK (1.76 ug/ml) for 0, 15, 30, and 60 minutes at 37OC. Samples were subjected than to SDS-PAGE, blotted and detected with anti SDF-1 polyclonal Ab or with the monoclonal Ab K15C, which specifically binds the amino terminal part of the chemokine.

### DESCRIPTION OF THE PREFERRED EMBODIMENTS

The invention relates to the use of a CTK inhibitor (CTKI) or CTK, a mutein, isoform, fused protein, functional derivative, active fraction, circularly permutated derivative, a salt or inducer thereof in the manufacture of a medicament for treating a disease, in which chemokine stromal cell-derived factor (SDF-1) is involved with the development and/or course of the disease.

The present invention is based on the finding that CTK is capable of specifically inhibiting SDF-1 activity. The inventors show herein that stress signals induce the following events: a transient increase in BM SDF-1, osteoclast activation, increase in CTK activity, decrease in BM SDF-1 concentration mediated by CTK degradation and cell mobilization.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The findings presented below demonstrate that stress signals, such as inflammation and injury, induce osteoclast activation and progenitor mobilization from the bone marrow into the peripheral blood (PB). The inventors show that stress signals induce the following events: a transient increase in BM SDF-1, osteoclast activation, increase in CTK activity, decrease in BM SDF-1 concentration mediated by CTK degradation and cell mobilization.

Also, the results obtained show that externally administrated SDF-1, or SDF-1 produced by the action of G-CSF and/or HGF, or stress signals (e.g. inflammation or injury) directly induces osteoclast activation, an increase in BM CTK, a decrease of SDF-1 and progenitor mobilization.

We demonstrated that RANKL (an inducer of osteoclast activation) administration in vivo induced mobilization which requires functional CXCR4 (SDF-1).

The results obtained in the present application are surprising since they show, in contrast to the results of Takamatsu et al. (Blood. 1998 Nov. 1; 92(9):3465-73. 1998), that osteoclast activation mediates stem/progenitor cell mobilization from the bone marrow to the circulation. It was demonstrated that CTK, the major osteoclast proteinase, inhibits SDF-1 activity by specific and fast cleavage of the amino terminus thereof.

Thus the present findings provide the possibility of using a cathepsin K inhibitor (CTKI) or CTK, a mutein, isoform, fused protein, functional derivative, active fraction, circularly permutated derivative, a salt or inducer thereof for modulating SDF-1 activity.

The development or course of many diseases involve up or down regulation of SDF-1 concentration and/or activity. Therefore, CTKI or CTK, a mutein, isoform, fused protein, functional derivative, active fraction, circularly permutated derivative, a salt or inducer thereof can be used in the manufacture of a medicament for treating a disease, wherein said disease is characterized by the involvement of SDF-1 with the development and/or course of the disease.

SDF-1 and its receptor, CXCR4, play important roles in human immunodeficiency virus type 1 (HIV-1) pathophysiology, leukocyte trafficking, inflammation, hematopoiesis, embryogenesis, angiogenesis, and cancer metastasis. Based on our findings, we suggest that CTK-mediated proteolysis of would inactivate and terminate SDF-1/CXCR4-dependent cell signaling.

In one aspect of the invention, CTK, a mutein, isoform, fused protein, functional derivative, active fraction, circularly permutated derivative, a salt or inducer thereof is used in a disease caused/aggravated by SDF-1 activity, such as tumor, infectious and inflammatory disease.

Increasing evidence implicates SDF-1/CXCR4 signaling system in the pathogenesis of tumors, infectious and inflammatory processes in several diseases such as allergic airway diseases Gonzalo et al., [J immunol 165:499-508, 2000], Lukacs et al., [Am. J Pathol. 160:1353-1360, 2002]), rheumatoid arthritis Buckley et al., [J immunol 165:3423-3429, 2000], and arteriosclerosis (Abi-Younes et al., [Circ Res 86:131-138, 2000], where SDF-1 is upregulated;

Abnormal expression of CXCR4 or SDF-1 has been observed in solid tumors such as prostate cancer (Taichman et al. Cancer Res 2002, 62:1832-7), kidney cancer (Br J Cancer 2002, 86:1250-6), neuroblastoma (J Immunol. 2001, 167:4747-57), glioma (Zhou et al. J Biol. Chem. 2002, 277:49481, Salmaggi et al J Neurooncol. 2004 May; 67(3):305-17.), pancreatic cancer (Clin. Cancer Res 2000, 6:3530), colon (Zeelenberg et al. Cancer Res 2003, 63:3833-9) and breast cancer (Muller et al. Nature 2001, 410:50-6).

Most leukemic cells respond to SDF-1 with increased adhesion, survival and proliferation (Juarez et al Histol Histopathol. 2004, 19 (1): 299-309).

SDF-1 is also involved in proliferation and survival of pre-BALL cells (Nishii et al. Br J Haematol. 1999 June; 105(3):701-10). Tavor et al. (Cancer Research 64, 2817-2824, 2004) have shown that acute myelogenous leukemia (AML) cells express SDF-1 and that blocking endogenous SDF-1 reduces AML cell survival.

SDF-1 in malignant glial tumors appears to have a role in angiogenesis and cross-talk between endothelial and tumoral cells. (Salmaggi et al J Neurooncol. 2004 May; 67(3):305-17). Thus inhibiting SDF-1 activity in such tumors with CTK, a mutein, isoform, fused protein, functional derivative, active fraction, circularly permutated derivative, a salt or inducer thereof is of clinical benefit.

SDF-1 and CXCR4 govern the metastatic destination of tumor cells. CXCR4 is expressed by human breast cancer cells and SDF-1 is high in organs that are the first destination of the metastasis in a mouse model, indicating that this chemokine signaling induces an invasive response (Müller et al., [Nature 410:50-56, 2001]). Thus blocking SDF-1 activity with CTK, a mutein, isoform, fused protein, functional derivative, active fraction, circularly permutated derivative, a salt or inducer thereof is beneficial for metastasis inhibition in tumor cell expressing CXC chemokine Receptor-4.

The results obtained demonstrate that, if desired, one can induce mobilization by inducing osteoclast activation and/or CTK activity. Alternatively, mobilization may be prevented by inhibiting osteoclast activation and/or CTK activity.

In a one embodiment of the invention, CTK, a mutein, isoform, fused protein, functional derivative, active fraction, circularly permutated derivative, a salt or inducer thereof is used for inducing mobilization of progenitors in severe neutropenia. For example, in severe neutropenia occurring after bone marrow transplantation and/or after intensive cancer chemotherapy.

In another embodiment of the invention, osteoclast activation is used for inducing mobilization of progenitors in severe neutropenia.

In another aspect of the invention, a CTKI is used in a disease which is prevented/alleviated by SDF-1 activity.

SDF-1 is known to be produced in a variety of tissues including lymphoid organs, liver, lung, and mesenchymal cells surrounding endothelial cells (Tachibana et al. 1998. Nature 393:591.).

CTK is relatively tissue specific, and is present in addition to osteoclasts, in macrophages, aortic smooth muscle cells, thyroid epithelial cells and lung epithelial cell. In organs populated with these cells CTKI are expected to act as SDF-1 agonists. For example, inhibition of the activity of CTK in the bone marrow will prevent SDF-1 degradation by this enzyme and subsequent mobilization.

Thus, in another embodiment, CTKI is used for inhibiting mobilization of progenitors and therefore for increase retention of progenitors in the bone marrow, for enhancing repopulation and for restoring defective hematopoiesis.

The stem cells/progenitors according to this aspect of the present invention are preferably obtained from the subject to be treated. However stem cells/progenitors may also be obtained from a syngeneic, allogeneic and less preferably from a xenogeneic donor.

It will be appreciated that when allogeneic or xenogeneic stem cells are used, the recipient subject and/or cells are preferably treated to prevent graft versus host and host versus graft rejections. Immunosuppression protocols are well known in the art and some are disclosed in U.S. Pat. No. 6,447,765.

CXCR4 can serve as coreceptor for T-cell tropic human immunodeficiency virus-1 (Oberlin et al., [Nature 382:833-835, 1996]). SDF-1, has been shown to compete with human immunodeficiency virus (HIV) for binding to CXCR4 by both occupying and promoting downregulation of this receptor; as such, it may play a role in host defense to this virus (Amara et al., [J Exp Med 186:1390-146, 1997]).

Thus in one embodiment of the invention, a CTKI is used in a disease which is prevented/alleviated by SDF-1 activity such as HIV.

In one aspect, the invention provides a method of treating a disease affected by up or down regulation of SDF-1 concentration and/or activity comprising effective amount of a CTKI or CTK, a mutein, isoform, fused protein, functional derivative, active fraction, circularly permutated derivative, a salt or inducer thereof.

In one embodiment, the invention provides a method of inhibiting metastasis of a tumor cell in a mammal, wherein the tumor cell expresses CXC Chemokine Receptor-4 (CXCR4), which method comprises administering to the mammal CTK in an amount sufficient to inhibit metastasis of the tumor.

In another aspect, the invention provides the use of CTKI, CTK, a mutein, isoform, fused protein, functional derivative, active fraction, circularly permutated derivative, a salt or inducer thereof, for the manufacture of a medicament for modulating targeting of pluripotent stem cells, such as normal progenitors, to tissues within a subject.

In one embodiment, the invention relates to increasing targeting of normal progenitor cells to the target tissue, such as bone marrow, comprising inhibiting CTK activity in the target tissue.

In another embodiment, the invention relates to the decrease of targeting of neoplastic cells to the target tissue, such as the bone marrow, comprising augmenting CTK activity in the target tissue.

CTK, a mutein, isoform, fused protein, functional derivative, active fraction, circularly permutated derivative, a salt or inducer thereof acts as SDF-1 inhibitor, thus, it will inhibit SDF-1 activity in leukocyte trafficking, inflammation, hematopoiesis, multiplication of hematopoietic cells, embryogenesis, angiogenesis, and cancer metastasis.

CTK inhibitor acts as SDF-1 agonist, thus, it enhances multiplication of hematopoietic cells in the bone marrow, in patients administered with an agent or treatment increasing BM SDF-1, such as e.g. irradiation or having a condition in which SDF-1 is increased in the bone marrow.

Thus, in one embodiment the invention relates to a method of increasing the rate of normal hematopoietic cell multiplication, comprising administering an effective amount of a CTK inhibitor to the hematopoietic cells.

In a preferred embodiment of the invention, the hematopoietic cells are selected from the group consisting of hematopoietic stem cells and hematopoietic progenitor cells.

In another preferred embodiment, said cells are in a patient and SDF-1 is administered or induced in the patient in need of such treatment.

In a further preferred embodiment, the patient in need of such treatment has cancer.

In a further preferred embodiment, the patient have need of autologous or allogeneic bone marrow or peripheral blood stem cell transplantation.

In another preferred embodiment, said patient is treated with a cytotoxic agent, wherein the effective amount of the CTK inhibitor is sufficient to reduce the susceptibility of the normal cells to the cytotoxic agent.

In one aspect of the invention, CTK inhibitor acts as SDF-1 agonist, thus, it enhances engraftment of hematopoietic cells to the bone marrow, in patients administered with an agent or treatment increasing BM SDF-1, such as e.g. irradiation or having a condition in which SDF-1 is increased in the bone marrow.

In one aspect of the invention, CTK acts as SDF-1 antagonist, thus, it mediates SDF-1 degradation leading to progenitor mobilization from the bone marrow into the peripheral blood.

According to one aspect of the present invention, there is provided a method of identifying a CTK antagonist comprising contacting CTK with SDF-1 and measuring the activity or integrity of SDF-1 and isolating a compound capable of preventing or reversing the inhibition of SDF-1 activity or degradation.

In one embodiment, the activity of SDF-1 measured is migration.

The present invention contemplates also the CTK antagonist obtainable by said method.

In another aspect, the invention relates to a method of treatment of a cancer patient in which CXCR4 is expressed in the cancer cells and/or SDF-1 is secreted by the cancer cells, the method comprising administration of a therapeutically effective amount of CTK to said patient.

In one embodiment of the invention, the type of cancer is one that typically metastasizes to an organ comprising cells expressing SDF-1.

In a further embodiment, the cancer typically metastasizes to the skin, liver, brain, and lung.

In a further embodiment, the tumor cell is selected from a lymphoma cell, a neuroblastoma cell, a lung cancer cell, an angiosarcoma cell, a leukemia cell, a glioma cell, or a melanoma cell, breast cancer cell or prostate cancer cell.

As used herein, the phrase “stem cells” refers to cells, which are capable of differentiating into other cell types having a particular, specialized function (i.e., “fully differentiated” cells).

As used herein a “transgenic cell” is a cell carrying an introduced gene or segment.

The term “mobilization” refers to a process in which cells are released from the bone marrow into the circulation due to imbalance of the steady state homeostasis, induced for example by the cytokine G-CSF.

The term “progenitor cells” refers to an heterogeneous population of immature undifferentiated hematopoietic cells, capable of colony formation. Progenitors are enriched for stem cells but they also include more mature cells that are incapable of long term bone marrow repopulation, which characterize true stem cells.

The term “TRAP+ MNC cells” relates to activated osteoclasts which are Multi Nucleated Cells expressing the enzyme TRAP (Tartrate Resistant Acid Phosphatase).

The term “stress signal” refers to an alarm situation induced by severe imbalance of the steady state conditions, accompanied by up or down regulation of cytokines, chemokines, proteolytic enzymes and adhesion molecules. Examples include inflammation, irradiation, toxic drugs, cytokine stimulation or deprivation.

The term “inhibitor of CTK” within the context of this invention refers to any molecule of downregulating CTK production, expression and/or action in such a way that CTK production and/or action is attenuated, reduced, or partially, substantially or completely prevented or blocked. The term “CTK inhibitor” is meant to encompass inhibitors of CTK production as well as of inhibitors of CTK action.

An inhibitor of production can be any molecule negatively affecting the synthesis, or processing CTK. The inhibitors considered according to the invention can be, for example, suppressors of gene expression of the CTK, antisense mRNAs reducing or preventing the transcription of the CTK mRNA or leading to degradation of the mRNA, proteins impairing correct folding, or partially or substantially preventing secretion of CTK, proteases degrading CTK and the like.

An inhibitor of CTK action can be an CTK antagonist, for example. Antagonists can either bind to or sequester the CTK molecule itself with sufficient affinity and specificity to partially or substantially neutralise the CTK or CTK binding site(s) responsible for CTK binding to its substrates.

As used herein the phrase “inhibiting an expression or activity” refers to partially or fully inhibiting expression (transcription and/or translation) or activity (e.g., enzymatic or ligand binding) of CTK. Several different approaches can be used to down regulate activity of CTK.

For example, inhibiting CTK activity can be achieved by an agent such as an antibody or an antibody fragment capable of specifically binding CTK. Preferably, the antibody specifically binds at least one epitope of CTK. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics. Preferred epitopes of CTK are those comprising the catalytic site or the region of association of CTK with SDF-1.

The term “antibody” as used herein includes intact molecules as well as functional fragments thereof, such as Fab, F(ab′)2, and Fv. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference). Commercially available polyclonal and monoclonal antibodies that bind to CTK, are suitable for use in the present invention.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (1972)]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (scFv) are prepared by constructing a structural gene comprising DNA sequences encoding the VH and VL domains connected by an oligonucleotide. The structural gene is inserted into an expression vector, which is subsequently introduced into a host cell such as E. coli.

A form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Inhibition of CTK activity can also be effected by utilizing known peptide inhibitors of CTK, or by any peptide inhibitor derived from a polypeptide sequence capable of interacting with the catalytic site of CTK (e.g., substrate analogue. Description of suitable biochemical/molecular approaches which can be utilized for identifying additional inhibitors is provided hereinbelow.

Additional inhibitors of CTK can be identified using molecular design approach, utilizing on the three-dimensional molecular structure of CTK described by Blanchard et al. (Structure 7:1125-1133, 1999) and by Watt et al. (Structure 7:1135-1143, 1999) and on its substrate binding model which has been created by Chou et al., (FEBS 419:49-54, 1997).

CTK activity can also be inhibited by a protein relocating CTK to a subcellular organelle/location and rendering it incapable of exerting its biological effect Downregulation of expression of CTK cells can be effected using any one of several molecular approaches.

For example, CTK transcription can be inhibited via RNA interference by utilizing a small interfering RNA (siRNA) molecule. RNA interference is a two step process; the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase (Hutvagner and Zamore, Curr. Opin. Genetics and Development 12:225-232, 2002).

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the CTK mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another agent capable of downregulating a CTK is a DNAzyme molecule capable of specifically cleaving an mRNA transcript or DNA sequence of the CTK. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Inhibition of CTK expression can also be effected by using an antisense polynucleotide capable of specifically hybridizing with an mRNA transcript encoding the CTK thereby specifically inhibiting translation of the CTK transcripts.

Design of antisense molecules which can be used to efficiently inhibit CTK expression must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft, J Mol Med 76: 75-6, 1998; Kronenwett et al., Blood 91: 852-62, 1998; Rajur et al., Bioconjug Chem 8: 935-40, 1997; Lavigne et al., Biochem Biophys Res Commun 237: 566-71, 1997; and Aoki et al., Biochem Biophys Res Commun 231: 540-5), 1997].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9, 1999].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16, 1374-1375, 1998).

Several clinical trials have demonstrated safety, feasibility and activity of antisense oligonucleotides. For example, antisense oligonucleotides suitable for the treatment of cancer have been successfully used (Holmund et al., Curr Opin Mol Ther 1:372-85, 1999), while treatment of hematological malignancies via antisense oligonucleotides targeting c-myb gene, p53 and Bcl-2 had entered clinical trials and had been shown to be tolerated by patients (Gerwitz Curr Opin Mol Ther 1:297-306, 1999).

More recently, antisense-mediated suppression of human heparanase gene expression has been reported to inhibit pleural dissemination of human cancer cells in a mouse model (Uno et al., Cancer Res 61:7855-60, 2001).

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

The antisense sequences may include a ribozyme sequence which is capable of cleaving transcripts encoding CTK, thereby preventing translational of those transcripts into functional CTK. Such a ribozyme is readily synthesizable using solid phase oligonucleotide synthesis.

Ribozymes are being increasingly used for the sequence-specific inhibition of gene expression by the cleavage of mRNAs encoding proteins of interest [Welch et al., “Expression of ribozymes in gene transfer systems to modulate target RNA levels.” Curr Opin Biotechnol. 1998 October; 9(5):486-96]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., “Ribozyme gene therapy for hepatitis C virus infection.” Clin Diagn Virol. 1998 Jul. 15; 10(2-3):163-71.]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Although the above describe expressible inhibitors (e.g., antibody fragments, antisense, etc.) can be synthesized using recombinant techniques and provided directly via, for example, injection, such molecules can also be expressed directly in cells by utilizing an expression vector which includes a polynucleotide sequence encoding the inhibitor positioned under the transcriptional control of a promoter sequence suitable for directing constitutive tissue specific or inducible transcription in mammalian cells.

Constitutive promoters suitable for use with the present invention include sequences which are functional (i.e., capable of directing transcription) under most environmental conditions and most types of cells such as the cytomegalovirus (CMV) and Rous sarcoma virus (RSV). Tissue specific promoters suitable for use with the present invention include sequences which are functional in hematopoietic cells, example include, for example, the promoter sequences described by Clark and Gordon (Leukoc Biol. 63:153-68, 1998); Stein et al. (Cancer 15::2899-902, 2000); and Hormas et al., (Curr Top. Microbiol. Immunol. 211:159-64, 1996). Inducible promoters suitable for use with the present invention include for example the tetracycline-inducible promoter (Srour et al., hromb. Haemost. 90: 398-405, 2003).

The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

Polyadenylation sequences can also be added to the expression vector in order to increase the translation efficiency of a polypeptide inhibitor such as Scfv. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can also be used by the present invention. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I).

Recombinant viral vectors are useful for in vivo expression of CTK inhibitors since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Further description of constructs which are suitable for hematopoeitic cell-specific expression is provided in Malik et al. (Blood 15: 86:2993-3005, 1995).

The use of a vector for inducing and/or enhancing the endogenous production of an endogenous inhibitor of CTK, in a cell normally silent for expression of an inhibitor, or expressing amounts of inhibitor which are not sufficient, are also contemplated according to the invention. The vector may comprise regulatory sequences functional in the cells desired to express the inhibitor. Such regulatory sequences comprise promoters or enhancers. The regulatory sequence is then introduced into the right locus of the genome by homologous recombination, thus operably linking the regulatory sequence with the gene, the expression of which is required to be induced or enhanced. The technology is usually referred to as “endogenous gene activation” (EGA), and it is described e.g. in WO 91/09955.

It will be understood by the person skilled in the art that it is also possible to shut down CTK expression using the same technique, i.e. by introducing a negative regulation element, like e.g. a silencing element, into the gene locus of CTK, which will result in down-regulation or prevention of CTK expression. The person skilled in the art will understand that such down-regulation or silencing of CTK expression has the same effect as the use of a CTKI in order to prevent and/or treat disease.

Various methods can be used to introduce the expression vector of the present invention into hematopoietic cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et al. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

It will be appreciated that the expression constructs utilized for expressing the inhibitor are preferably constructed and introduced into hematopoietic cells in a manner which enables exclusive and controllable expression in these cells. For example, by utilizing a viral expression vector which can exclusively transform hematopoeitc cells or by transforming such cells ex-vivo, and by utilizing an inducible promoter sequence in the expression construct (see examples above), exclusive and controllable expression in these cells can be achieved.

Such an expression strategy is advantageous in particularly when used in context of leukemia treatment, since it allows for precise control over hematopoesis and thus regulation of hematopoeitic cell numbers.

As is mentioned hereinabove, downregulation of CTK expression or activity may be effected in vitro by exposing cultured hematopoietic cells to a downregulating agent, or in vivo by administering such an agent to a subject. These in vivo and in vitro approaches can be utilized to treat

Non limiting examples of CTK inhibitors: peptide inhibitors such as Mu-Leu-hph-us-ph (Palmer J Med. Chem 1995, 38:3193-3196), non-peptide inhibitors such as SB-462795, SB-357114, 462795 (Glaxo Swithkline's), E64, AAR494, anti sense (Inui et al J Biol Chem. 1997 Mar. 28; 272(13):8109-12.), NH-linked aryl/heteroaryl CTKI (Robichaud et al Bioorg Med Chem Lett. 2004 Aug. 16; 14(16):4291-5), AAE581 (Novartis).

CTK inhibitors are also disclosed in:: WO00/55126 and WO01/49288 Taveres et al (J Med Chem 47:5049, 2004, J med Chem 29:47(3):588-99, 2004), Catalano et al (Bioorg Med Chem Lett. 2004 9; 14(3):719-22, and 14(1) 275-8), Robichaud et al. (J Med Chem 14; 46(17):3709-27, 2003), Setti et al. (Bioorg Med Chem Lett 13(12):2051-3, 2003), Altmann et al. (Bioorg Med Chem Lett 13(12):1997-2001, 2003), Chen et al. J Org Chem, 68(7):2633-8, 2003).

The terms “polypeptide and protein” in the present specification are interchangeable.

The present invention also concerns muteins of CTK protein of the invention, which muteins retain essentially the same biological activity of the CTK protein having essentially only the naturally occurring sequences of the CTK. Such “muteins” may be ones in which up to about 20 and 10 amino acid residues may be deleted, added or substituted by others in the CTK protein respectively, such that modifications of this kind do not substantially change the biological activity of the protein mutein with respect to the protein itself.

These muteins are prepared by known synthesis and/or by site-directed mutagenesis techniques, or any other known technique suitable thereof.

Any such mutein preferably has a sequence of amino acids sufficiently duplicative of that of the basic the CTK such as to have substantially similar activity thereto. Thus, it can be determined whether any given mutein has substantially the same activity as the basic protein of the invention by means of routine experimentation comprising subjecting such a mutein to the biological activity tests set forth in Examples below.

Muteins of the CTK protein which can be used in accordance with the present invention, or nucleic acid coding thereof, include a finite set of substantially the CTK corresponding sequences as substitution peptides or polynucleotides which can be routinely obtained by one of ordinary skill in the art, without undue experimentation, based on the teachings and guidance presented herein. For a detailed description of protein chemistry and structure, see Schulz, G. E. et al., Principles of Protein Structure, Springer-Verlag, New York, 1978; and Creighton, T. E., Proteins: Structure and Molecular Properties, W.H. Freeman & Co., San Francisco, 1983, which are hereby incorporated by reference. For a presentation of nucleotide sequence substitutions, such as codon preferences, see. See Ausubel et al., Current Protocols in Molecular Biology, Greene Publications and Wiley Interscience, New York, N.Y., 1987-1995; Sambrook et al., Molecular Cloning: A Laboratory Manual, Cold Spring Harbor Laboratory, Cold Spring Harbor, N.Y., 1989.

Preferred changes for muteins in accordance with the present invention are what are known as “conservative” substitutions. Conservative amino acid substitutions of those in the protein having essentially the naturally-occurring CTK sequences, may include synonymous amino acids within a group, which have sufficiently similar physicochemical properties that substitution between members of the group will preserve the biological function of the molecule, see Grantham, Science, Vol. 185, pp. 862-864 (1974). It is clear that insertions and deletions of amino acids may also be made in the above-defined sequence without altering its function, particularly if the insertions or deletions only involve a few amino acids, e.g., under 50, and preferably under 20 CTK and do not remove or displace amino acids which are critical to a functional conformation, e.g., cysteine residues, Anfinsen, “Principles That Govern The Folding of Protein Chains”, Science, Vol. 181, pp. 223-230 (1973). Muteins produced by such deletions and/or insertions come within the purview of the present invention. Preferably, the synonymous amino acid groups are those defined in Table A. More preferably, the synonymous amino acid groups are those defined in Table B; and most preferably the synonymous amino acid groups are those defined in Table C.

TABLE A
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE B
More Preferred Groups of Synonymous Amino Acids

| Amino Acid | Synonymous Group |
|---|---|
| Sers | Sers |
| Arc | His, Lys, Arg |
| Leu | Ile, Phe, Met, Leu |
| Pro | Ala, Pro |
| Thr | Thr |
| Ala | Pro, Ala |
| Val | Met, Ile, Val |
| Gly | Gly |
| Ilea | Ile, Met, Phe, Val, Leu |
| Phe | Met, Tyr, Ile, Leu, Phe |
| Try | Phi, Try |
| Cys | Ser, Cys |
| His | Arg, Gln, His |
| Gln | Glu, His, Gln |
| Asn | Asp, Asn |
| Lys | Arg, Lys |
| Asp | Asn, Asp |
| Glu | FLN, Glu |
| Met | Phe, Ile, Val, Leu, Met |
| Trp | Trp |

TABLE C
Most Preferred Groups of Synonymous Amino Acids

| Amino Acid | Synonymous Group |
|---|---|
| Sers | Sers |
| Arc | Arc |
| Leu | Ile, Met, Leu |
| Pro | Pro |
| Thr | Thar |
| Alan | Alan |
| Val | Val |
| Gly | Gly |
| Ilea | Ile, Met, Leu |
| Phi | Phi |
| Try | Tyr |
| Cys | Ser, Cys |
| His | His |
| Gln | Gln |
| Asn | Asn |
| Lys | Lys |
| Asp | Asp |
| Glu | Glu |
| Met | Ile, Leu, Met |
| Trp | Trp |

Examples of production of amino acid substitutions in proteins which can be used for obtaining muteins of the protein for use in the present invention include any known method steps, such as presented in U.S. Pat. Nos. RE 33,653, 4,959,314, 4,588,585 and 4,737,462, to Mark et al; 5,116,943 to Koths et al., 4,965,195 to Namen et al; 4,879,111 to Chong et al; and 5,017,691 to Lee et al; and lysine substituted proteins presented in U.S. Pat. No. 4,904,584 (Straw et al).

In another preferred embodiment of the present invention, any mutein of the CTK protein for use in the present invention has an amino acid sequence essentially corresponding to that of the above noted CTK protein of the invention. The term “essentially corresponding to” is intended to comprehend muteins with minor changes to the sequence of the basic protein which do not affect the basic characteristics thereof, particularly insofar as its ability to the CTK is concerned. The type of changes which are generally considered to fall within the “essentially corresponding to” language are those which would result from conventional mutagenesis techniques of the DNA encoding the CTK protein of the invention, resulting in a few minor modifications, and screening for the desired activity for example increasing the sensitivity of stem cells to a chemoattractant.

The present invention also encompasses CTK variants. A preferred CTK variant are the ones having at least 80% amino acid identity, a more preferred the CTK variant is one having at least 90% identity and a most preferred variant is one having at least 95% identity to CTK amino acid sequence.

The term “sequence identity” as used herein means that the amino acid sequences are compared by alignment according to Hanks and Quinn (1991) with a refinement of low homology regions using the Clustal-X program, which is the Windows interface for the ClustalW multiple sequence alignment program (Thompson et al., 1994). The Clustal-X program is available over the internet at ftp://ftp-igbmc.u-strasbg.fr/pub/clustalx/. Of course, it should be understood that if this link becomes inactive, those of ordinary skill in the art could find versions of this program at other links using standard internet search techniques without undue experimentation. Unless otherwise specified, the most recent version of any program referred herein, as of the effective filing date of the present application, is the one, which is used in order to practice the present invention.

Another method for determining “sequence identity” is the following. The sequences are aligned using Version 9 of the Genetic Computing Group's GDAP (global alignment program), using the default (BLOSUM62) matrix (values −4 to +11) with a gap open penalty of −12 (for the first null of a gap) and a gap extension penalty of −4 (per each additional consecutive null in the gap). After alignment, percentage identity is calculated by expressing the number of matches as a percentage of the number of amino acids in the claimed sequence.

Muteins in accordance with the present invention include those encoded by a nucleic acid, such as DNA or RNA, which hybridizes to DNA or RNA under stringent conditions and which encodes a the CTK protein in accordance with the present invention, comprising essentially all of the naturally-occurring sequences encoding the CTK and sequences which may differ in its nucleotide sequence from the naturally-derived nucleotide sequence by virtue of the degeneracy of the genetic code, i.e., a somewhat different nucleic acid sequence may still code for the same amino acid sequence, due to this degeneracy.

The term “hybridization” as used herein shall include any process by which a strand of nucleic acid joins with complementary strand through a base pairing (Coombs J, 1994, Dictionary of Biotechnology, stokton Press, New York N.Y.). “Amplification” is defined as the production of additional copies of a nucleic acid sequence and is generally carried out using polymerase chain reaction technologies well known in the art (Dieffenbach and Dveksler, 1995, PCR Primer, a Laboratory Manual, Cold Spring Harbor Press, Plainview N.Y.).

“Stringency” typically occurs in a range from about Tm-5° C. (5° C. below the melting temperature of the probe) to about 20° C. to 25° C. below Tm.

The term “stringent conditions” refers to hybridization and subsequent washing conditions, which those of ordinary skill in the art conventionally refer to as “stringent”. See Ausubel et al., Current Protocols in Molecular Biology, Greene Publications and Wiley Interscience, New York, N.Y., 1987-1995; Sambrook et al., Molecular Cloning: A Laboratory Manual, Cold Spring Harbor Laboratory, Cold Spring Harbor, N.Y., 1989.

As used herein, stringency conditions are a function of the temperature used in the hybridization experiment, the molarity of the monovalent cations and the percentage of formamide in the hybridization solution. To determine the degree of stringency involved with any given set of conditions, one first uses the equation of Meinkoth et al. (1984) for determining the stability of hybrids of 100% identity expressed as melting temperature Tm of the DNA-DNA hybrid:

Tm=81.5 C+16.6 (Log M)+0.41 (% GC)−0.61 (% form)−500/L

where M is the molarity of monovalent cations, % GC is the percentage of G and C nucleotides in the DNA, % form is the percentage of formamide in the hybridization solution, and L is the length of the hybrid in base pairs. For each 1 C that the Tm is reduced from that calculated for a 100% identity hybrid, the amount of mismatch permitted is increased by about 1%. Thus, if the Tm used for any given hybridization experiment at the specified salt and formamide concentrations is 10 C below the Tm calculated for a 100% hybrid according to the equation of Meinkoth, hybridization will occur even if there is up to about 10% mismatch.

As used herein, “highly stringent conditions” are those which provide a Tm which is not more than 10 C below the Tm that would exist for a perfect duplex with the target sequence, either as calculated by the above formula or as actually measured. “Moderately stringent conditions” are those, which provide a Tm, which is not more than 20 C below the Tm that would exist for a perfect duplex with the target sequence, either as calculated by the above formula or as actually measured. Without limitation, examples of highly stringent (5-10 C below the calculated or measured Tm of the hybrid) and moderately stringent (15-20 C below the calculated or measured Tm of the hybrid) conditions use a wash solution of 2×SSC (standard saline citrate) and 0.5% SDS (sodium dodecyl sulfate) at the appropriate temperature below the calculated Tm of the hybrid. The ultimate stringency of the conditions is primarily due to the washing conditions, particularly if the hybridization conditions used are those, which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE (standard saline-phosphate-EDTA), 5×Denhardt's reagent, 0.5% SDS, 100 &micro; g/ml denatured, fragmented salmon sperm DNA at a temperature approximately 20 to 25 C below the Tm. If mixed probes are used, it is preferable to use tetramethyl ammonium chloride (TMAC) instead of SSC (Ausubel, 1987, 1999). Adult stem cells can be obtained using a surgical procedure such as bone marrow aspiration or can be harvested using commercial systems such as those available from Nexell Therapeutics Inc. Irvine, Calif., USA. Stem cells utilized by the present invention are preferably collected (i.e., harvested) using a stem cell mobilization procedure, which utilizes chemotherapy or cytokine stimulation to release of HSCs into circulation of subjects. Stem cells are preferably retrieved using this procedure since mobilization is known to yield more HSCs and progenitor cells than bone marrow surgery.

“Isoforms” of CTK are proteins capable of degrading SDF-1 or fragment thereof which may be produced by alternative splicing.

The term “circularly permuted derivatives” as used herein refers to a linear molecule in which the termini have been joined together, either directly or through a linker, to produce a circular molecule, and then the circular molecule is opened at another location to produce a new linear molecule with termini different from the termini in the original molecule. Circular permutations include those molecules whose structure is equivalent to a molecule that has been circularized and then opened. Thus, a circularly permuted molecule may be synthesized de novo as a linear molecule and never go through a circularization and opening step. The preparation of circularly permutated derivatives is described in WO95/27732.

In yet a further embodiment, the substance according to the invention comprises an immunoglobulin fusion, i.e. the molecules according to the invention are fused to all or a portion of an immunoglobulin. Methods for making immunoglobulin fusion proteins are well known in the art, such as the ones described in WO 01/03737, for example. The person skilled in the art will understand that the resulting fusion protein of the invention retains the biological activity of the CTK. The resulting fusion protein ideally has improved properties, such as an extended residence time in body fluids (half-life), increased specific activity, increased expression level, or facilitated purification of the fusion protein.

Preferably, the substance according to the invention is fused to the constant region of an Ig molecule. It may be fused to heavy chain regions, like the CH2 and CH3 domains of human IgG1, for example. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero- or homomultimeric.

Functional derivatives of the substance according to the invention may be conjugated to polymers in order to improve the properties of the protein, such as the stability, half-life, bioavailability, tolerance by the human body, or immunogenicity.

Therefore, a preferred embodiment of the invention relates to a functional derivative of the substance according to the invention comprising at least one moiety attached to one or more functional groups which occur as one or more side chains on the amino acid residues.

A highly preferred embodiment relates to a substance of the invention linked to Polyethlyenglycol (PEG). PEGylation may be carried out by known methods, such as the ones described in WO 92/13095, for example.

An “active fraction” according to the present invention may e.g. be a fragment of CTK. The term fragment refers to any subset of the molecule, that is, a shorter peptide which retains the desired biological activity. Fragments may readily be prepared by removing amino acids from either end of the CTK molecule and testing the resultant fragment for its properties to degrade SDF-1. Proteases for removing one amino acid at a time from either the N-terminal or the C-terminal of a polypeptide are known, and so determining fragments which retain the desired biological activity involves only routine experimentation.

As active fractions of an CTK, muteins and fused proteins thereof, the present invention further covers any fragment or precursors of the polypeptide chain of the protein molecule alone or together with associated molecules or residues linked thereto, e.g., sugar or phosphate residues, or aggregates of the protein molecule or the sugar residues by themselves, provided said fraction has substantially similar activity to CTK e.g. degrades SDF-1.

The term “salts” herein refers to both salts of carboxyl groups and to acid addition salts of amino groups of the CTK molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of CTK, e.g. the ability to degrade SDF-1.

Stem cell mobilization can be induced with CTK or osteoclast activation alone or in combination to a number of molecules. Examples include but are not limited to cytokines such as, granulocyte colony-stimulating factor (G-CSF), granulocyte-macrophage colony-stimulating factor (GM-CSF), interleukin (IL)-7, IL-3, IL-12, stem cell factor (SCF), and flt-3 ligand; chemokines like IL-8, Mip-1α, Groβ, or SDF-1; and the chemotherapeutic agents cyclophosphamide (Cy) and paclitaxel. It will be appreciated that these molecules differ in kinetics and efficacy, however, according to presently known embodiments G-CSF is preferably used alone or in combination such as with cyclophosphamide to mobilize the stem cells. Typically, G-CSF is administered daily at a dose of 5-10 μg/kg for 5-10 days. Methods of mobilizing stem cells are disclosed in U.S. Pat. Nos. 6,447,766 and 6,162,427. Human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium, which enables its outgrowth. Following 9 to 15 days, the ICM derived outgrowth is dissociated into clumps either by a mechanical dissociation or by enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 1-2 weeks. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; Gardner et al., [Fertil. Steril. 69: 84, 1998].

It will be appreciated that commercially available stem cells can be also be used according to this aspect of the present invention. Human ES cells can be purchased from the NIH human embryonic stem cells registry (<http://escr.nih.gov>). Non-limiting examples of commercially available embryonic stem cell lines are BG01, BG02, BG03, BG04, CY12, CY30, CY92, CY10, TE03, and TE32.

Human EG cells can be retrieved from the primordial germ cells obtained from human fetuses of about 8-11 weeks of gestation using laboratory techniques known to anyone skilled in the arts. The genital ridges are dissociated and cut into small chunks, which are thereafter disaggregated into cells by mechanical dissociation. The EG cells are then grown in tissue culture flasks with the appropriate medium. The cells are cultured with daily replacement of medium until cell morphology consistent with EG cells is observed, typically after 7-30 days or 1-4 passages. For additional details on methods of preparing EG cells see Shamblott et al., [Proc. Natl. Acad. Sci. USA 95: 13726, 1998] and U.S. Pat. No. 6,090,622.

It will be appreciated that enrichment of stem cell population exhibiting pluripotency may be preferably effected. Thus, for example, as outlined hereinabove, CD34+ stem cells can be concentrated using affinity columns or FACS as further described hereinunder.

Culturing of stem cells under proliferative conditions may also be effected in cases where stem cell numbers are too low for use in treatment. Culturing of stem cells is described in U.S. Pat. Nos. 6,511,958, 6,436,704, 6,280,718, 6,258,597, 6,184,035, 6,132,708 and 5,837,5739.

Analysis of CXCR4 receptor level for example in tumor cells can be detected by flow cytometry. This approach employs instrumentation that scans single cells flowing past excitation sources in a liquid medium. The technology can provide rapid, quantitative, multiparameter analyses on single living (or dead) cells based on the measurement of visible and fluorescent light emission. This basic protocol focuses on: measure fluorescence intensity produced by fluorescent-labeled antibodies and ligands that bind specific cell-associated molecules. To isolate cell populations using fluorescence activated cell sorter stem cells of the present invention are contacted with anti CXCR4 commercially available from R&D, 614 McKinley Place NE Minneapolis, Minn.

Other cytological or biochemical methods for quantitatively assessing the level of the chemotactic receptor expression include but are not limited to binding analysis using a labeled (e.g., radioactively labeled) chemokine, western blot analysis, cell-surface biotinylation and immunofluorescent staining. It will be appreciated that the receptor expression levels can also be determined at the mRNA level. For example, CXCR4 mRNA may be detected in cells by hybridization to a specific probe. Such probes may be cloned DNAs or fragments thereof, RNA, typically made by in-vitro transcription, or oligonucleotide probes, usually generated by solid phase synthesis. Methods for generating and using probes suitable for specific hybridization are well known and used in the art. Quantification of mRNA levels can be also effected using an amplification reaction [e.g., PCR, “PCR Protocols: A Guide To Methods And Applications”, Academic Press, San Diego, Calif. (1990)], employing primers, which hybridize specifically to the mRNA of a chemotactic receptor of interest.

A variety of controls may be usefully employed to improve accuracy in mRNA detection assays. For instance, samples may be hybridized to an irrelevant probe and treated with RNAse A prior to hybridization, to assess false hybridization.

Functional assays can also be used to determine the chemotactic receptor expression. For example, a chemotaxis assay, which employs a gradient of the chemotactic agent (e.g., SDF-1) and follows stem cell migration through a membrane towards the chemotactic agent can be utilized to identify and isolate stem cells exhibiting increased chemotaxis. If the cells do not express enough levels of the chemotactic receptor (e.g., CXCR4), then the majority of the cells will remain on the membrane. However, upon increased expression of the chemoattractant receptor of the present invention, cells will migrate through the membrane and settle on the bottom of the well of the chemotaxis plate (see Example 3 of the Examples section) It will be appreciated that a functional homing assay can also be utilized by the method of the present invention. Such an assay is described in Kollet (2001) Blood 97:3283-3291 (ref 12).

Preferred individual subjects according to the present invention are mammals such as canines, felines, ovines, porcines, equines, bovines and preferably humans.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A Laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### LPS-Induced Inflammation Leads to Osteoclast Activation

Bone destruction is a pathological hallmark of several chronic inflammatory diseases including rheumatoid arthritis and periodontitis. Inflammation-induced bone loss of this sort results from osteoclast activation and induction of elevated numbers of bone-resorbing osteoclasts.

We explored the effect of inflammation induced by endotoxin lipopolysaccharide (LPS) administration, on osteoclast activation in the bone marrow (BM).

LPS (Sigma) was administered to Balb/c mice in a single subcutaneous injection of 250 μg/mouse, and saline was injected instead of LPS in control groups. 5 days following LPS administration, mice were sacrificed, bones were fixed, decalcified, paraffin embedded and sectioned, BM harvested, and the level of TRAP+ osteoclast (activated osteoclast) monitored.

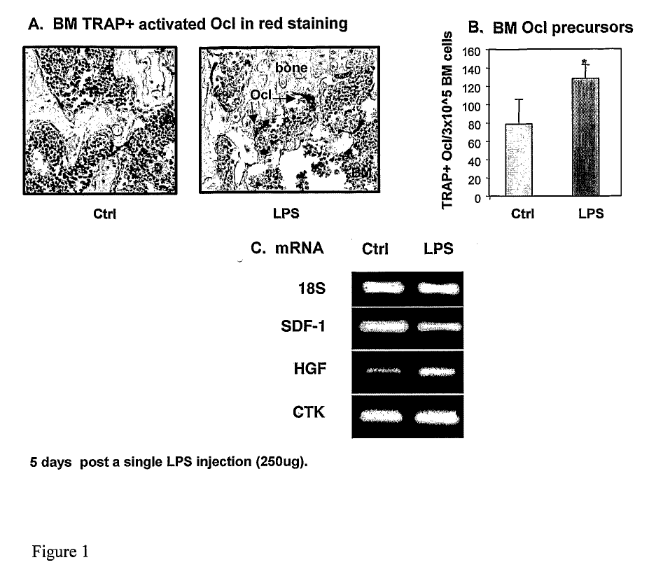

We found a dramatic increase in TRAP+ osteoclasts (as detected by red staining using the kit for TRAP staining, produced by Sigma). In this assay substrate is exogeneously provided to measure intrinsic activity of the tissue TRAP in the BM of LPS treated mice (FIGS. 1A and B).

Next, we explored the effect of LPS mediated inflammation on transcription of bone marrow mediators such as SDF-1, hepatocyte growth factor (HGF) and osteoclast specific protease CTK.

One femur of each mouse was flushed with Trireagent for RNA extraction. 1 ug RNA was used for the preparation of cDNA. Semi quantitative RT-PCR was used to measure the transcription of mouse SDF-1, HGF and CTK. The following primers were used: SDF-1 (Ponomaryov, 2000, J. Clin. Invest. 106:1331): left primer (SEQ ID NO: 1) 5′-GGACGCCAAGGTCGTCGCCGTG-3′, right primer (SEQ ID NO: 2) 5′-TTGCATCTCCCACGGATGTCAG-3′. HGF (Weinstein, 2001, Moll Cell boil, 21(15):5122): left primer (SEQ ID NO: 3) 5′-TGCCAGAAAGATATCCCGAC-3′, right primer (SEQ ID NO: 4) 5′-AACTCGGATGTTTGGGTCAG-3′. CTK: left primer (SEQ ID NO: 5) 5′-GGCCTCTCTTGGTGTCCATA-3′, right primer (SEQ ID NO: 6) 5′-TCTGCTGCACGTATTGGAAG-3′.

We observed a dramatic increase in HGF and CTK transcription and a decrease in SDF-1 transcription in the BM of LPS treated mice (FIG. 1C).

Thus, the results obtained demonstrate that LPS induced inflammation (by 5 day treatment) mediates the following changes in the bone marrow: increase in osteoclast activation, increase of CTK and HGF transcription and a decrease in SDF-1 transcription.

### Example 2

### LPS-Induced Inflammation Leads to Decrease of BM SDF-1 Concentration and Progenitor Mobilization

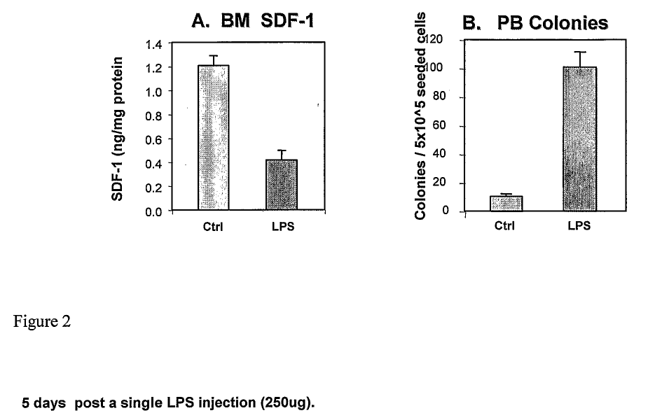

In the previous example we showed that inflammation mediates decrease of SDF-1 transcription in the bone marrow. We carried out the following experiments to explore whether, in addition, there is a decrease of SDF-1 concentration in the BM following LPS mediated inflammation. Thus, we monitored SDF-1 concentration in the BM in LPS treated mice versus untreated mice (FIG. 2A).

Mice were administered for 5 days with LPS as described in example 1 and SDF-1 concentration in bone marrow was monitored (by ELISA as described by Petit et al 2002).

The results summarized in FIG. 2A show that LPS mediated inflammation (by 5 days treatment) induces a decrease in BM SDF-1 concentration (of about 66%), from 1.2 in non-treated control group to 0.4 ng/mg protein in LPS treated group).

Since mobilization of progenitors from the BM to the circulation by the well known mobilizing agent granulocyte colony-stimulating factor (G-CSF), is preceded by a decrease on BM SDF-1 concentration (Petit et al Nature immunology (3) N0 7 687, 2002), we explored whether LPS mediated inflammation and SDF-1 decrease in the BM, is followed by mobilization (FIG. 2B).

Mice were administered for 5 days with LPS (as described in Example 1) and progenitor increase in peripheral blood (PB) was measured (as in Example 9).

We found in PB of LPS treated mice (for 5 days) increased levels of progenitors (about 95% higher) compared to the level of progenitors in PB of control non-treated mice (FIG. 2B). For example, about 100 progenitor colonies per 5×10⁵ seeded cells were found in LPS and control groups versus 5 colonies per 5×10⁵ seeded cells in non treated groups.

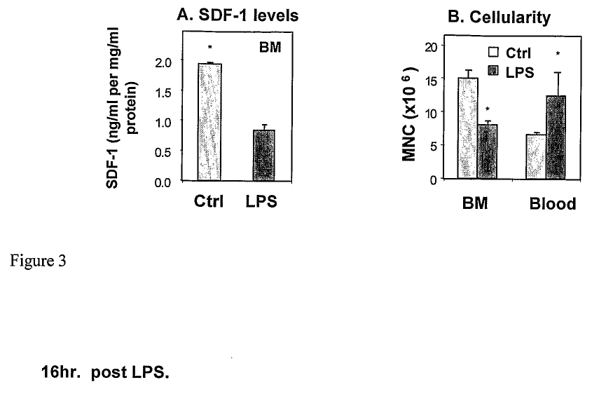

In order to verify that the progenitor increase detected in PB in the LPS treated mice, results from the egress or mobilization of cells from the bone marrow, mononuclear cells (MNC) were monitored in the BM and in PB by counting numbers of ficolled WBC (leukocytes), using hemacytometer shortly after LPS treatment (16 hours post LPS treatment) and in BM and PB of non treated mice (FIG. 3).

We found that LPS mediated inflammation induces decrease in BM SDF-1 concentration (about 55%), decrease of MNC cells in the BM (about 53%) and increase of MNC cells in the PB (about 78%) when compared to the respective non treated mice (FIG. 3).

Thus, 16 hours post LPS administration, a decrease in BM SDF-1 concentration and egress of cells from the BM to the PB is observed.

### Example 3

### Functional CXCR4 is Required for LPS-Mediated Mobilization

G-CSF is known to induce stem cell mobilization by decreasing BM SDF-1 and up-regulating CXCR4 (Petit et al 2002). We carried out experiments in order to determine whether LPS mediated mobilization of progenitors requires CXCR4/SDF-1 interaction.

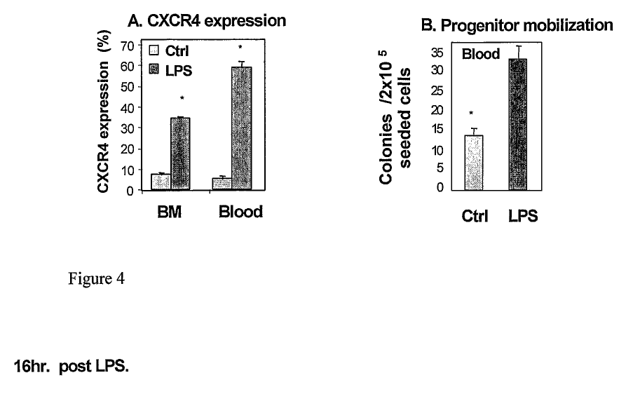

We monitored CXCR4 expression in BM and PB of LPS treated mice, 16 hours post LPS administration, and in control non treated mice. We found a significant increase on the level of CXCR4 expression as detected by flow cytometry (Petit 2002) in both BM and PB of LPS treated mice (FIG. 4A) which correlated with a significant increase in progenitor mobilization (FIG. 4B).

The results indicate that mobilization of progenitor cells by LPS involve SDF-1/CXCR4 interactions.

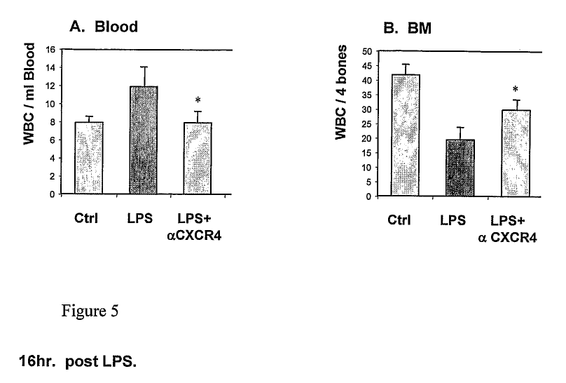

To confirm that functional CXCR4 is required for LPS mediated mobilization, mice were treated with LPS alone (16 hours), co-treated with LPS and anti CXCR4 antibody (anti rat CXCR4, which is also effective on murine CXCR4, Torrey Pines Biolabs, CA, 10 mcg in 500 mcl PBS), or remained untreated, and white blood cells (WBC) in the PB or in the BM were monitored by using hemacytometer 16 hours post treatment (FIG. 5).

We found that LPS mediated mobilization is reduced in the absence of functional CXCR4 (FIG. 5). Thus, the results confirm that functional CXCR4 and CXCR4/SDF-1 interaction are necessary for LPS mediated mobilization.

Therefore, like in mobilization induced by G-CSF (Petit et al 2002), LPS mediated mobilization involves increase in CXCR4 and decrease of SDF-1 in the bone marrow.

The decrease of the SDF-1 in the bone marrow following LPS mediated mobilization is probably due to SDF-1 degradation.

According to the results shown in examples 1-3, inflammation induces BM osteoclast activation, CTK and HGF expression, SDF-1 reduction and progenitor mobilization. It was also shown that inflammation mediated mobilization, like G-CSF mediated mobilization, involves BM SDF-1 reduction and requires functional CXCR4.

### Example 4

### Injury Leads to Mobilization

We carried out experiments to explore cell mobilization in a model of injury induced by controlled bleeding (a single bleeding of 1% of body weight).

The following parameters were monitored after induction of bleeding:

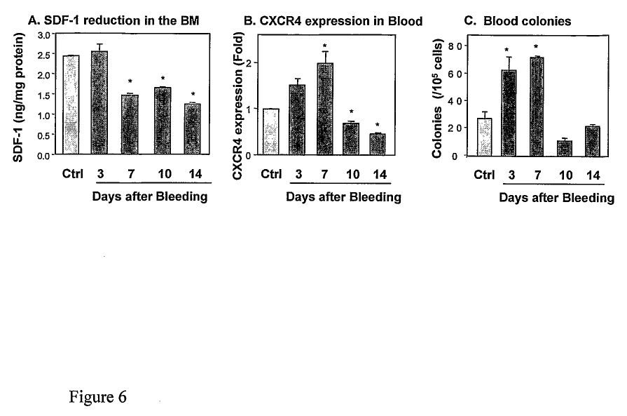

A—mobilization of progenitors, B—SDF-1 levels in bone marrow, and C—CXCR4 expression in the blood (FIG. 6).

We observed significant and stable reduction in BM SDF-1 concentration from day 7 to 14 after bleeding initiation. A transient increase in CXCR4 expression was seen at days 3 to 7 after bleeding (FIG. 6). A transient increase in progenitors in PB was detected at days 3 to 7 after bleeding (FIG. 6).

Therefore, injury caused by controlled bleeding induces a stable decrease in BM SDF-1 concentration, and a transient increase in PB CXCR4 and progenitor mobilization.

### Example 5

### Injury Affects Osteoclast Activation

We examined the effect of injury, induced by controlled bleeding, on osteoclast activation in the BM.

The experimental setting included induction of controlled bleeding (as in Example 4) and monitoring the activation marker of osteoclast, TRAP+ in the BM (as in Example 1) in treated versus untreated mice.

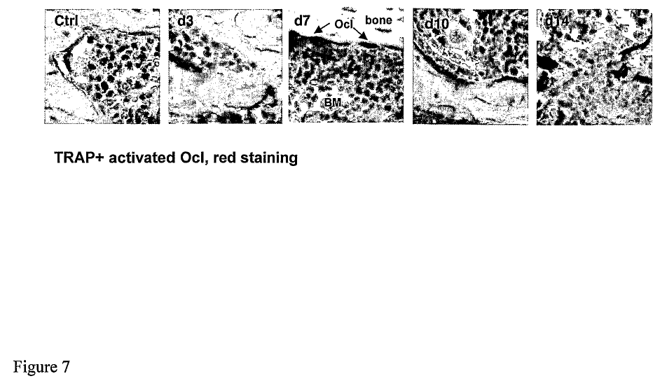

We could detected dramatic osteoclast activation within the BM at day 7 after bleeding induction (FIG. 7).

The above findings in Examples 1-5 demonstrate that stress signals, such as inflammation and injury, induce progenitor mobilization from the bone marrow into the PB. Involved in triggering of stress signal mobilization, are a decrease in BM SDF-1 concentration, and increase in CXCR4 expression in the BM, as in G-CSF mediated mobilization. Unexpectedly, we have found that stress-signals mediated mobilization involves osteoclast activation.

### Example 6

### Factors that Mediate Osteoclast Activation

We carried out experiments to explore in vitro whether mediators of cell mobilization and motility such as G-CSF, SDF-1 and HGF (Lapidot 2002 Exp Hematol, SDF-1, Aiuti 1997, J Exp Med, and Kollet et al JCI 112:160-169 (2003).) are capable of inducing osteoclast activation.

Osteoclast precursors are stimulated by cell-cell contact with osteoblasts to become activated multinucleated TRAP+ bone resorbing cells. To obtain active osteoclasts in vitro, a clvarian osteoblasts are incubated with Vitamine D3 and PG2E which potentiate their M-CSF production and RANKL expression, needed for osteoclast activation. BM cells which contain osteoclast precursors are seeded then on the osteoblast monolayer, for 5 days, to obtain active multinucleated osteoclasts.

A co-culture of primary mouse osteoblasts and BM (containing ocl precursors as detailed above) was seeded in the presence of G-CSF (50 ng/ml), HGF (50 ng/ml), or SDF-1 (10 and 100 ng/ml). Five days later, TRAP+ multinucleated osteoclasts in the cultures were stained (as described in Example 1) and counted 5 days later.

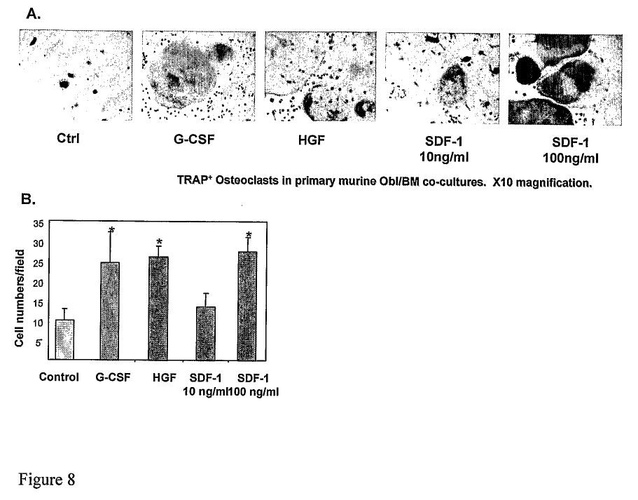

We observed that treatment with either of the mediators G-CSF, HGF and SDF-1 significantly increased the level of activated TRAP+ osteoclasts (FIG. 8A representative and 8B summary).

We carried out further experiments to explore which one of the above mediators directly activates osteoclasts.

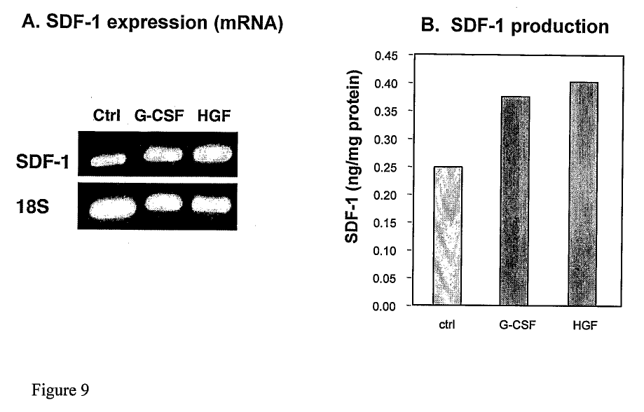

Osteoblast were incubated with G-CSF, HGF or remained untreated, and SDF-1 transcription and expression was determined in such cells (FIG. 9).

Primary calvaria osteoblasts were cultured for 3 days in the presence of SDF-1 (PeproTech, 100 ng/nl), HGF (PeproTech, 50 ng/ml) and G-CSF (Filgrastin, Roche, 50 ng/ml). Conditioned media was collected to determine SDF-1 concentration by ELISA, as described in Example 2. Determination of SDF-1 transcription and expression as in Example 2.

We showed that both, G-CSF and HGF induce SDF-1 transcription and SDF-1 production in osteoblasts (FIGS. 9A and 9B respectively), thus SDF-1 appears to be the factor that is induced in osteoblasts by G-CSF and/or HGF action and which directly induces osteoclast activation.

We stained culture of primary murine osteoclast and found that osteoclast express CXCR4 (not shown).

The results obtained show that G-CSF and HGF induce SDF-1 expression in osteoblasts and that the SDF-1 binds CXCR4 on the osteoclast surface and leads to osteoclast activation. We hypotheses that SDF-1 mediated osteoclast activation induces progenitor mobilization.

We carried out the following experiment to verify in vivo our hypothesis that SDF-1 induces osteoclast activation and hematopoietic progenitor cell (HPC) mobilization. Balb/c mice were injected subcutaneously for five consecutive days with 10 mcg SDF-1 or 1.5 mcg HGF or both, and osteoclast activation (measured by TRAP+ staining as in Example 1) and mobilization to the peripheral blood, (assayed in semi solid culture as in Example 1) was determined.

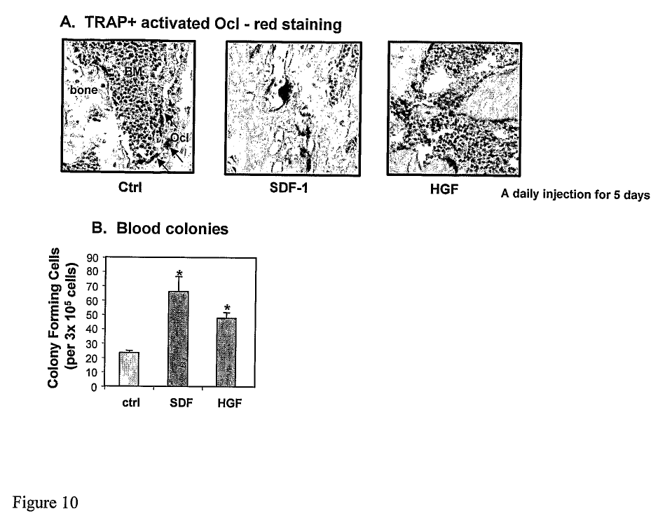

We found that SDF-1 and HGF administration in vivo induced both osteoclast activation (FIG. 10A) and mobilization of progenitors (FIG. 10B).

In all, the results obtained show that externally administrated SDF-1, or SDF-1 produced by the action of G-CSF and/or HGF, or stress signals (e.g. inflammation or injury) directly induce osteoclast activation and progenitor mobilization.

### Example 7

### Direct Osteoclast Activation by Receptor Activator of NF-KappaB Ligand (RANKL) Induces In-Vivo Hematopoietic Stem Cell Mobilization

RANKL is an osteoclast differentiating factor (SUDA et al Endocr Rev 20:345, 1999). We tested whether activation of osteoclast by RANKL induces,

A—expression of proteases typically secreted by activated osteoclast such as CTK and MMP-9 in the bone marrow (Delaisse, Clin Chim Acta Feb 15; 291(2):223-34 2002), and
B—progenitor mobilization.

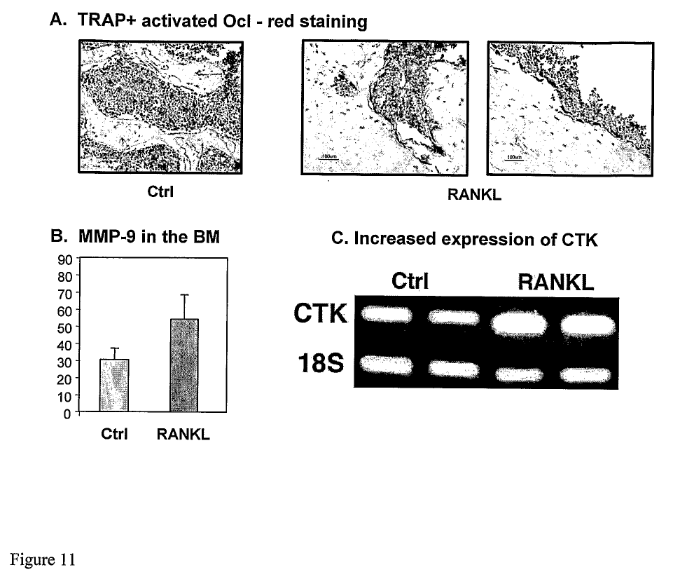

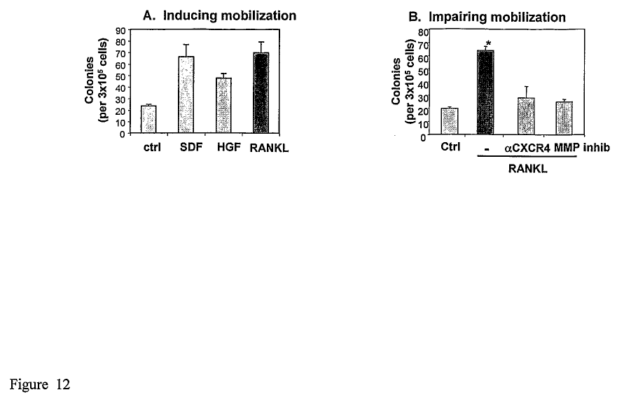

Mice were administrated with RANKL as follows: 2 daily subcutaneous injections of 5 mcg for 3 days and after two more days in the absence of RANKL. The following parameters were monitored in both treated and control untreated mice: TRAP+ activated osteoclasts (FIG. 11A), induction of osteoclast proteases MMP-9 (FIG. 11B) and CTK (FIG. 11C) in the BM and mobilization (FIG. 12).

We observed that RANKL administration induced formation of TRAP+ active osteoclasts along the endosteum (BM region linking the bone, known to contain stem and progenitor cells), induction of MMP-9 and CTK expression in the bone marrow (FIGS. 11A, B and C respectively) and mobilization of progenitors (FIG. 12 A). Impaired mobilization was found following treatment with RANKL together with an MMP inhibitor or with anti CXCR4 neutralizing antibodies (FIG. 12B). Thus, mobilization by osteoclast activation, induced by RANKL, requires CXCR4/SDF-1 interactions and MMP 2/9 function (FIG. 12 B).

The above demonstrates that RANKL administration in vivo induced osteoclast activation and mobilization which requires functional CXCR4/SDF-1 interactions.

### Example 8

### Mechanism of Mobilization by Osteoclast Activation

We demonstrated in the previous examples that mobilization by stress signals involves transient SDF-1 production by BM osteoblast, such produced SDF-1 binds surface osteoclasts CXCR4 leading to their activation. Osteoclast activation in turn triggers SDF-1 degradation and BM SDF-1 concentration decrease.

In view of the results obtained in Example 1, we hypothesized that specific SDF-1 degradation is induced by the major osteoclast bone resorbing enzyme, CTK. Therefore, we explored the effect of CTK on the activity of SDF-1.

We employed an in-vitro functional assay for SDF-1 (Example 10) to check the effect of CTK on SDF-1 activity. The assay consisted on migration of leukemic Pre B ALL G2 (cells which migrate very well to relative low concentrations of SDF-1, Spiegel, Blood, 2004) to SDF-1.

In brief, SDF-1 (125 ng) was incubated with CTK (ug/ml) in a 100 ul reaction volume adjusted with PBS, for about 16 hours in 37° C. In some samples, protease inhibitor (PI, 1 ul, Sigma, containing the cystein protease inhibitor E-64) was preincubated with CTK for 1.5 hrs, in 37° C., before SDF-1 was added. The leukemic Pre B ALL G2 cells were loaded on transwells and migration towards CTK treated as well as non treated SDF-1 was measured.

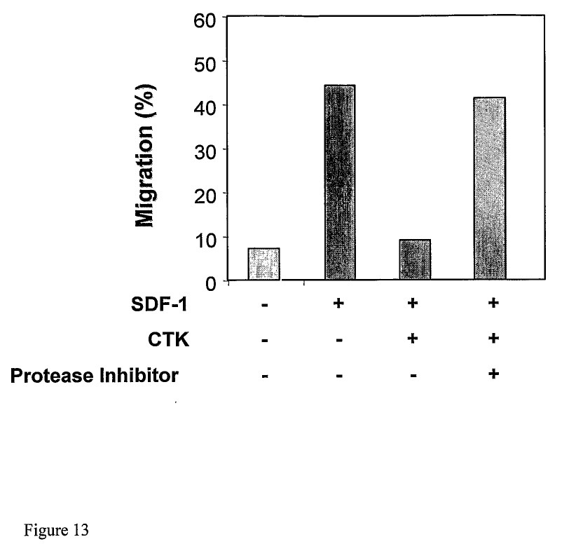

We found that the chemotactic activity of SDF-1 was abolished by CTK, but not in the presence of the protease inhibitor (PI) (FIG. 13).

The results obtained and shown in FIG. 13 demonstrate that CTK inhibits SDF-1 activity.

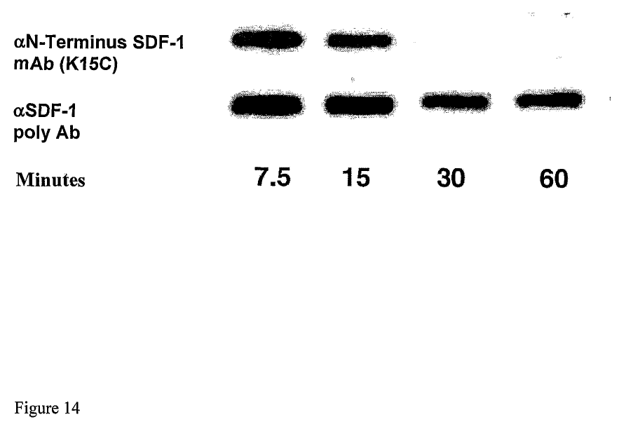

We further explored the kinetics of SDF-1 cleavage by CTK at a time scale of minutes (FIG. 14). Samples of SDF-1 (20 ng) were incubated with CTK (1.76 ug/ml) for 0, 15, 30, 60, 120 and 240 minutes at 37° C. After collection, the samples were subjected to SDS-PAGE and blotted and detected with anti SDF-1 polyclonal Ab (R&D) or with the monoclonal Ab K15C which specifically binds the amino terminal part of the chemokine.

The results obtained show fast and site specific SDF-1 cleavage by CTK.

Thus it was demonstrated that CTK, the major osteoclast proteinase, inhibits SDF-1 activity by specific and fast cleavage of the amino terminus thereof.

### Example 9

### CFU Assay

In order to detect the levels of mouse progenitors in the blood circulation, semisolid cultures were performed as previously described (Petit, Nat Immunol, 2002). In brief, mouse BM cells (3×10⁵ cells/ml) were plated in 0.9% methylcellulose (Sigma), 30% FCS, 5×10⁻⁵M 2ME, 50 ng/ml SCF, 5 ng/ml IL-3, 5 ng/ml GM-CSF (R&D), and 2 u/ml erythropoietin (Orto Bio Tech, Don Mills, Canada). The cultures were incubated at 37° C. in a humidified atmosphere containing 5% CO₂ and scored 7 days later by inverted microscopy by morphologic criteria.

### Example 10

### Migration Assay

125 ng/ml SDF-1α was kept untreated or incubated with CTK (1 mcg/ml) for overnight in 37° C. To inhibit enzymatic activity of CTK, 1 and 2 mcl of protease inhibitor (Sigma) was incubated with CTK for 1.5 h before adding to SDF-1. RPMI (600 μl) supplemented with 10% FCS was added with tested SDF-1 to the lower chamber of a Costar 24-wells transwell (Corning (pore size 5 μm), NY). 1×10⁵ pre B ALL G2 cells in 100 μl medium were loaded to the upper chamber and were allowed to migrate for 4 hours at 37° C. Migrating cells were collected from the lower chamber and counted for 60 seconds using a FACSCalibur. Control spontaneous migration was performed without SDF-1α in the lower chamber.

### Example 11

### Animals

Eight-ten week old Balb/c mice were purchased from Harlan Rehovot. All the experiments were approved by the animal care committee of the Weizmann Institute.

### REFERENCES

1. Broxmeyer, H. E., 1983, CRC Critical Review in Oncology/Hematology 1:227-257)
2. Kim and Broxmeyer, [Blood 91:100-110, 1998]
3. Peled et al., [J Clin Invest. 1999 November; 104(9):1199-211. 1999]).
4. Nagasawa et al.,: Semin Immunol. 1998 June; 10(3):179-85. [1998]
5. Suzuki et al., The Journal of Immunology, 2001, 167: 3064-3073.
6. Valenzuela-Fernandez et al., J Biol Chem. 2002 May 3; 277(18):15677-89.
7. Bromme, D., and Okamoto, K. (1995) Biol. Chem. Hoppe-Seyler 376, 379-384.
8. Drake, F. H., Dodds, R. A., James, I. E., Connor, J. R., Debouck, C., Richardson, S., Lee-Rykaczewski, E., Coleman, L., Rieman, D., Barthlow, R., Hastings, G., and Gowen, M. (1996) J. Biol. Chem. 271, 12511-12516).
9. Brömme, D., Okamoto, K., Wang, B. B., and Biroc, S. (1996) J. Biol. Chem. 271, 2126-2132.
10. Nagasawa T, Hirota S, Tachibana K, Takakura N, Nishikawa S, Kitamura Y, Yoshida N, Kikutani H, Kishimoto T. Defects of B-cell lymphopoiesis and bone-marrow myelopoiesis in mice lacking the CXC chemokine PBSF/SDF-1. Nature. 1996; 382:635-638
11. Ma Q, Jones D, Borghesani P R, Segal R A, Nagasawa T, Kishimoto T, Bronson R T, Springer T A. Impaired B-lymphopoiesis, myelopoiesis, and derailed cerebellar neuron migration in CXCR4− and SDF-1-deficient mice. Proc Natl Acad Sci USA. 1998; 95:9448-9453
12. Kollet O, Spiegel A, Peled A, Petit I, Byk T, Hershkoviz R, Guetta E, Barkai G, Nagler A, Lapidot T. Rapid and efficient homing of human CD34(+)CD38(−/low)CXCR4(+) stem and progenitor cells to the bone marrow and spleen of NOD/SCID and NOD/SCID/B2m(null) mice. Blood. 2001; 97:3283-3291
13. Peled A, Petit I, Kollet O, Magid M, Ponomaryov T, Byk T, Nagler A, Ben-Hur H, Many A, Shultz L, Lider O, Alon R, Zipori D, Lapidot T. Dependence of human stem cell engraftment and repopulation of NOD/SCID mice on CXCR4. Science. 1999; 283:845-848
14. Ponomaryov T, Peled A, Petit I, Taichman R, Habler L, Sandbank J, Arenzana-Seisdedos F, Magerus A, Caruz A, Fujii N, Nagler A, Lahav M, Szyper-Kravitz M, Zipori D, Lapidot T. Induction of the chemokine stromal-derived factor-1 following DNA damage improves human stem cell function. JCI. 2000; 106:1331-1339
15. Kollet O, Petit I, Kahn J, Samira S, Dar A, Peled A, Deutsch V, Gunetti M, Piacibello W, Nagler A, Lapidot T. Human CD34+CXCR4− sorted cells harbor intracellular CXCR4, which can be functionally expressed and provide NOD/SCID repopulation. Blood. 2002; 100
16. Spencer A, Jackson J, Baulch-Brown C. Enumeration of bone marrow ‘homing’ haemopoietic stem cells from G-CSF-mobilised normal donors and influence on engraftment following allogeneic transplantation. Bone Marrow Transplant. 2001; 28:1019-1022.
17. Voermans C, Kooi M L, Rodenhuis S, van der Lelie H, van der Schoot C E, Gerritsen W R. In vitro migratory capacity of CD34+ cells is related to hematopoietic recovery after autologous stem cell transplantation. Blood. 2001; 97:799-804
18. Lapidot T, Kollet O. The essential roles of the chemokine SDF-1 and its receptor CXCR4 in human stem cell homing and repopulation of transplanted immune-deficient NOD/SCID and NOD/SCID/B2m(null) mice. Leukemia. 2002; 16: 1992-2003
19. Forster R, Kremmer E, Schubel A, Breitfeld D, Kleinschmidt A, Nerl C, Bernhardt G, Lipp M. Intracellular and surface expression of the HIV-1 coreceptor CXCR4/fusin on various leukocyte subsets: rapid internalization and recycling upon activation. J Immunol. 1998; 160:1522-1531
20. Kollet O, Shivtiel S, Chen Y Q, Suriawinata J, Thung S N, Dabeva M D, Kahn J, Spiegel A, Dar A, Samira S, Goichberg P, Kalinkovich A, Arenzana-Seisdedos F, Nagler A, Hardan I, Revel M, Shafritz D A, Lapidot T. HGF, SDF-1, and MMP-9 are involved in stress-induced human CD34+ stem cell recruitment to the liver. J Clin Invest. 2003; 12:160-169
21. Rusten L, Cue L, Pharo A, Jacobsen S, Lapidot T, Kvalheim G. TNF-a and TGF-b potently upregulate the expression of CXCR4 on peripheral blood progenitor cells. Blood. 2000; 94:252a
22. Denning-Kendall P, Singha S, Bradley B, Hows J. Cytokine expansion culture of cord blood Cd34+ cells induces marked and sustained changes in adhesion receptor and CXCR4 expressions. Stem Cells. 2003; 21:61-70
23. Bhatia M, Wang J C Y, Kapp U, Bonnet D, Dick J E. Purification of primitive human hematopoietic cells capable of repopulating immune-deficient mice. Proc Natl Acad Sci USA. 1997; 94:5320-5325
24. Wright D E, Bowman E P, Wagers A J, Butcher E C, Weissman I L. Hematopoietic stem cells are uniquely selective in their migratory response to chemokines. J Exp Med. 2002; 195:1145-1154
25. Grafte-Faure S, Levesque C, Ketata E, Jean P, Vasse M, Soria C, Vannier J P. Recruitment of primitive peripheral blood cells: synergism of interleukin 12 with interleukin 6 and stromal cell-derived FACTOR-1. Cytokine. 2000; 12:1-7
26. Broxmeyer H E, Hangoc G, Cooper S, H. K C. Enhanced myelopoiesis in sdf-1-transgenic mice: sdf-1 modulates myelopoeisis by regulating progenitor cell survival and inhibitory effects of myelosuppresive chemokines [abstract]. Blood. 1999; 94:650a
27. Lataillade J J, Clay D, Dupuy C, Rigal S, Jasmin C, Bourin P, Le Bousse-Kerdiles M C. Chemokine SDF-1 enhances circulating CD34(+) cell proliferation in synergy with cytokines: possible role in progenitor survival. Blood. 2000; 95:756-768
28. Lataillade J J, Clay D, Bourin P, Herodin F, Dupuy C, Jasmin C, Bousse-Kerdiles M C. Stromal cell-derived factor 1 regulates primitive hematopoiesis by suppressing apoptosis and by promoting G(0)/G(1) transition in CD34(+) cells: evidence for an autocrine/paracrine mechanism. Blood. 2002; 99:1117-1129.
29. Broxmeyer H, Kohli L, Kim C, Lee Y, Mantel C, Cooper S, Hangoc G, Shaheen M, Li X, Clapp D. Stromal cell-derived factor-1/CXCL12 directly enhances survival/antiapoptosis of myeloid progenitor cells through CXCR4 and Gai proteins and enhances engraftment of competitive, repopulating stem cells. J. Leukoc. Biol. 2003; 73:630-638
30. Cashman J, Clark-Lewis I, Eaves A, Eaves C. Stromal-derived factor 1 inhibits the cycling of very primitive human hematopoietic cells in vitro and in NOD/SCID mice. Blood. 2002; 99:792-799.
31. Cashman J, Dykstra B, Clark-Lewis I, Eaves A, Eaves C. Changes in the proliferative activity of human hematopoietic stem cells in NOD/SCID mice and enhancement of their transplantibility after in vivo treatment with cell cycle inhibitors. J. Exp. Med. 2002; 196:1141-1149
32. Ma Q. Jones D, Springer T A. The chemokine receptor CXCR4 is required for the retention of B lineage and granulocytic precursors within the bone marrow microenvironment. Immunity. 1999; 10:463-471
33. Kawabata K, Ujikawa M, Egawa T, kawamoto H, Tachibana K, lizasa H, Katsura Y, kishimoto T, Nagasawa T. A cell-autonomous requirement for CXCR4 in long-term lymphoid and myeloid reconstitution. Proc. Natl. Acad. Sci. USA. 1999; 96:5663-5667
34. Yahata T, Ando K, Sato T, Miyatake H, Nakamura Y, Mugurumu Y, Kato S, Hotta T. A highly sensitive strategy for SCID-repopulating cell assay by direct injection of primitive human hematopoietic cells into NOD/SCID mice bone marrow. Blood. 2003; 101:2905-2913
35. Wang J, Kimura T, Asada R, Harada S, Yokota S, Kawamota Y, Fujimura Y, Tsuji T, Ikehara S, Sonoda Y. SCID-repopulating cell activity of human cord blood-derived CD34− cells assured by intra-bone marrow injection. Blood. 2003; 101:2924-2931
36. Shen H, Cheng T, Olszak I, Garcia-Zepeda E, Lu Z, Herrmann S, Fallon R, Luster A D, Scadden D T. CXCR-4 desensitization is associated with tissue localization of hemopoietic progenitor cells. J Immunol. 2001; 166:5027-5033
37. Sweeney E A, Lortat-Jacob H, Priestley G V, Nakamoto B, Papayannopoulou T. Sulfated polysaccharides increase plasma levels of SDF-1 in monkeys and mice: involvement in mobilization of stem/progenitor cells. Blood. 2002; 99:44-51
38. Levesque J-P, Bendall L J, Hendy J, Williams B, Simmons P J. SDF-1a is inactivated by proteolytic cleavage in the bone marrow of mice mobilized by either G-CSF or cyclophosphamide. Blood. 2001; 98:831a
39. Moore M A, Hattori K, Heissig B, Shieh J H, Dias S, Crystal R G, Rafli S. Mobilization of endothelial and hematopoietic stem and progenitor cells by adenovector-mediated elevation of serum levels of SDF-1, VEGF, and angiopoietin-1. Ann. N.Y. Acad. Sci. 2001; 938:36-45, 45-37
40. Hattori K, Heissig B, Tashiro K, Honjo T, Tateno M, Shieh J H, Hackett N R, Quitoriano M S, Crystal R G, Rafii S, Moore M A. Plasma elevation of stromal-derived factor-1 induces mobilization of mature and immature hematopoietic progenitor and stem cells. Blood. 2001; 97:3354-3360
41. Petit I, Szyper-Kravitz M, Nagler A, Lahav M, Peled A, Habler L, Ponomaryov T, Taichman R S, Arenzana-Seisdedos F, Fujii N, Sandbank J, Zipori D, Lapidot T. G-CSF induces stem cell mobilization by decreasing bone marrow SDF-1 and up-regulating CXCR4. Nat. Immunol. 2002; 3:687-694
42. Sawada S, Gowrishankar K, Kitamura R, Suzuki M, Suzuki G, Tahara S, Koito A. Disturbed CD4+ T cell homeostasis and in vitro HIV-1 susceptibility in transgenic mice expressing T cell line-tropic HIV-1 receptors. J. Exp. Med. 1998; 187:1439-1449
43. Guenechea G, Gan O I, Inamitsu T, Dorrell C, Pereira D, Kelly M, Naldini L, Dick J. Transduction of human CD34+CD38− bone marrow and cord blood-derived SCID-repopulating cells with third generation lentiviral vectors. Mol. Ther. 2000; 1:566-573
44. Miyoshi H, Smith K, Mosier D, Verma I, Torbett B. Transduction of human CD34+ cells that mediate long-term engraftment of NOD/SCID mice by HIV vectors. Science. 1999; 283:682-686
45. Barquinero J, Segovia J, Ramirez M, Limon A, Guenechea G, Puig T, Briones J, Garcia J, Bueren J. Efficient transduction of human hematopoietic repopulating cells generating stable engraftment of transgene-expressing cells in NOD/SCID mice. ‘blood. 2000:3085-3093
46. Woods N, Fahlman C, Mikkola H, Hamiaguchi I, Olsson K, Zufferey R, Jacobsen S, Trono D, Karlsson S. Lentiviral gene transfer into primary and secondary NOD/SCID repopulating cells. Blood. 2000; 96:3725-3733
47. Sutton R, Reitsma M, Uchida N, Brown P. Transduction of human progenitor hematopoietic stem cells by human immunodeficiency virus type 1-based vectors is cell cycle dependent. J Virol. 1999; 73:3649-3660
48. Cavazzana-Calvo M, Hacein-Bey S, Basile G. Gene therapy of human severe combined immunodeficiency (SCID)-X1 disease. Science. 2000; 288:669-672
49. Aiuti A, Slavin S, Aker M, Ficara F, Deola S, Morteellaro A, Morecki S, Andolfi G, Tabucchi A, Carlucci F, Marinello E, Cattaneo F, Vai S, Servida P, Miniero R, Roncarolo M, Bordignon C. Correction of ADA-SCID by stem cell gene therapy combined with nonmyeloblative conditioning. Science. 2002; 296:2410-2413
50. Darash-Yahana M, Kahn J, Aslan H, Gropp M, Nagler A, Gazit Z, Reubinoff B, Lapidot T, Gazit D, Galun E, Peled A. Rapid and efficient lentiviral mediated transduction of human mesenchymal and hematopooietic stem cells. Submitted. 2003
51. Wagstaff M, Lilley C, Smith J, Robinson M, Coffin R, Latchman D. Gene transfer using a disabled herpes virus vector containing the EMCV IRES allows multiple gene expression in vitro and in vivo. Gene Ther. 1998; 5:1566-1570
52. Metcalf D. Haemopoietic colonies: In vitro cloning of normal and leukemic cells. Recnt Results in Cancer Res. 1977; 61:1
53. Gibellini D, Bassini A, Re M C, Ponti C, Miscia S, Gonelli A, La Placa M, Zauli G. Stroma-derived factor 1 alpha induces a selective inhibition of human erythroid development via the functional upregulation of Fas/CD95 ligand. Br J Haematol. 2000; 111:432-440
54. Bleul C C, Aiuti A, Fuhlbrigge R C, Casasnovas J M, Springer T A. A highly efficacious lymphocyte chemoattractant, stromal cell-derived factor 1 (SDF-1). The Journal of Experimental Medicine. 1996; 184:1101-1109
55. Signoret N, Oldbridge J, Perchen-Matthews A, Klasse P J, Tran T, Brass L F, Rosenkilde M M, Schwartz T W, Holmes W, Dallas W, Luther M A, Wells T N, Hoxie J A, Marsh M. Phorbol esters and SDF-1 induce rapid endocytosis and down modulation of the chemokine receptor CXCR4. J. Cell Biol. 1997; 139:651-664

1. A method of treating a disease in a subject wherein the development and/or course of said disease involves SDF-1 activity and/or concentration, said method comprising administering to the subject a cathepsin K inhibitor (CTKI) or CTK, a mutein, isoform, fused protein, functional derivative, active fraction, circularly permutated derivative, a salt or inducer thereof.

2. The method of claim 1, wherein the disease is caused/aggravated by SDF-1 activity.

3. The method of claim 2, wherein the disease is selected from cancer, inflammation and infection.

4. The method of claim 3, wherein the disease is allergic airway disease.

5. The method of claim 3, wherein the disease is rheumatoid arthritis.

6. The method of claim 3, wherein the disease is arteriosclerosis.

7. The method of claim 3, wherein the disease is cancer.

8. The method of claim 7, wherein the disease is selected from the group consisting of prostate cancer, kidney cancer, neuroblastoma, glioma, pancreatic cancer, colon cancer, breast cancer, leukemia.

9. (canceled)

10. (canceled)

11. (canceled)

12. (canceled)

13. (canceled)

14. (canceled)

15. The method of claim 7, wherein said administration prevents or reduces preventing metastasis.

16. The use according to claim 15, wherein the cancer cell expresses CXCR4.

17. (canceled)

18. The method of claim 8, wherein the leukemia is acute lymphoblastic leukemia (ALL).

19. The method of claim 8, wherein the leukemia is Acute Myeloid Leukemia (AML).

20. The method of claim 1, wherein the disease is prevented/alleviated by SDF-1 activity.

21. The method of claim 20, wherein the disease is HIV.

22. A method of inducing mobilization of stem cells in a subject in need thereof, comprising administering to said subject a composition comprising a CTK, a mutein, isoform, fused protein, functional derivative, active fraction, circularly permutated derivative, a salt or inducer in a pharmaceutically acceptable carrier.

23. The method of claim 22, wherein the subject in need suffers from severe neutropenia.

24. The method of claim 23, wherein neutropenia occurs upon bone marrow transplantation.

25. The method of claim 23, wherein neutropenia occurs upon cancer chemotherapy.

26. A method of increasing retention of stem cells in the bone marrow in a subject in need thereof, comprising administering to said subject CTK1.

27. The method of claim 26, wherein said administration enhances repopulation of an organ in a subject in said subject.

28. The method of claim 27, wherein the organ is the bone marrow.

29. A method of treating a disease which its development and course is affected by SDF-1 activity and/or concentration, comprising administration of an effective amount of CTKI or a CTK, a mutein, isoform, fused protein, functional derivative, active fraction, circularly permutated derivative, a salt or inducer thereof.

30. A method of treating cancer in a mammal, comprising administering to the mammal an effective amount of CTK, a mutein, isoform, fused protein, functional derivative, active fraction, circularly permutated derivative, a salt or inducer thereof.

31. A method according to claim 29, wherein the cancer cell expresses CXC Chemokine Receptor-4 (CXCR4).

32. A method according to claim 30, wherein said administering prevents metastasis.

33. A method of modulating targeting of pluripotent stem cells to tissues comprising the administration of an effective amount of a CTKI or CTK, a mutein, isoform, fused protein, functional derivative, active fraction, circularly permutated derivative, a salt or inducer thereof in a subject in need.

34. A method according to claim 33, wherein a CTKI is administered to a target tissue for increasing targeting of the cells to the target tissue.

35. The method of claim 34, wherein the cells are normal hematopoietic cells.

36. The method according to claim 35, wherein the hematopoietic cells is selected from the group consisting of hematopoietic stem cells and hematopoietic progenitor cells.

37. The method according to claim 36, wherein the cells are in vivo in a patient and a therapeutically effective amount of the CXCR4 agonist is administered to the patient in need of such treatment.

38. The method according to claim 36, wherein the CXCR4 agonist is SDF-1, a mutein, isoform, fused protein, functional derivative, active fraction, circularly permutated derivative, a salt or inducer thereof.

39. The method according to claim 38, wherein the patient has a cancer.

40. The method according to claim 39, wherein the patient requires autologous or allogeneic bone marrow or peripheral blood stem cell transplantation.

41. The method of claim 40, further comprising treating the patient with a cytotoxic agent.

42. A method according to claim 33, wherein a CTK, a mutein, isoform, fused protein, functional derivative, active fraction, circularly permutated derivative, a salt or inducer thereof is administered to a target tissue for reducing targeting of the cells to the target tissue.

43. The method according to claim 42, wherein the cells are neoplastic cells.

44. A method of reducing the rate of hematopoietic cell multiplication, comprising administering an effective amount of a CTK, a mutein, isoform, fused protein, functional derivative, active fraction, circularly permutated derivative, a salt or inducer thereof to the hematopoietic cells.

45. The method according to claim 44, wherein the hematopoietic cells is selected from the group consisting of hematopoietic stem cells and hematopoietic progenitor cells.

46. The method according to claim 45, wherein the cells are in vivo in a patient and a therapeutically effective amount of the CXCR4 agonist is administered to the patient in need of such treatment.

47. The method according to claim 46, wherein the patient requires autologous or allogeneic bone marrow or peripheral blood stem cell transplantation.

48. The method according to claim 47, wherein the patient has a cancer.

49. The method of claim 48, further comprising treating the patient with a cytotoxic agent.

50. A method of identifying a CTK antagonist comprising contacting CTK with SDF-1, measuring the activity of SDF-1 and isolating a compound capable of preventing or reversing inhibition of SDF-1 activity by CTK.

51. A method of identifying a CTK antagonist comprising contacting CTK with SDF-1, checking the integrity of SDF-1 and isolating a compound capable of preventing the degradation SDF-1 activity by CTK.

52. An antagonist obtained by the method according to claim 43.

53. An antagonist obtained by the method according to claim 44.


SEQUENCE LISTING (6 sequences)

SEQ ID NO: 1 - DNA, 22 bp, Artificial sequence
  PRIMER SDF-1
ggacgccaaggtcgtcgccgtg

SEQ ID NO: 2 - DNA, 22 bp, artificial sequence
  PRIMER SDF-1
ttgcatctcccacggatgtcag

SEQ ID NO: 3 - DNA, 20 bp, artificial sequence
  PRIMER HGF
tgccagaaagatatcccgac

SEQ ID NO: 4 - DNA, 20 bp, artificial sequence
  PRIMER HGF
aactcggatgtttgggtcag

SEQ ID NO: 5 - DNA, 20 bp, artificial sequence
  PRIMER CTK
ggcctctcttggtgtccata

SEQ ID NO: 6 - DNA, 20 bp, artificial sequence
  PRIMER CTK
tctgctgcacgtattggaag